(19) United States
(12) Patent Application Publication
(10) Pub. No.: US 2020/0352441 A1
(43) Pub. Date: Nov. 12, 2020

(54) Efficient Monitoring, Recording, and Analyzing of Physiological Signals

(71) Applicants: Orhan Soykan, Lino Lakes, MN (US); Quan Ni, Shoreview, MN (US); Mark Alan Christopherson, Shoreview, MN (US)

(72) Inventors: Orhan Soykan, Lino Lakes, MN (US); Quan Ni, Shoreview, MN (US); Mark Alan Christopherson, Shoreview, MN (US)

(21) Appl. No.: 16/406,042

(22) Filed: May 8, 2019

(51) Int. Cl. A61B 5/00; A61B 5/042; A61B 5/053; A61B 5/145; A61B 7/02

(52) U.S. Cl. CPC A61B 5/0028; A61B 5/686; A61B 5/0031; A61B 5/0015; A61B 5/746; A61B 2562/0219; A61B 5/0538; A61B 5/14542; A61B 7/023; A61B 2560/0481; A61B 5/042

(57) ABSTRACT

A novel implantable device to reliably record and transmit physiological signals from a subject was described. Design of the device to capture the signals, compress them, transmit via multiple techniques and to interpret the data are shown. Furthermore, the design and operation of the device in a typical setting are taught and the resulting implementation is presented.

### TECHNICAL FIELD

This document generally describes devices, systems, and methods for monitoring, recording, and analyzing physiological signals.

### BACKGROUND

Cardiac electrical signals (e.g., an electrocardiogram (“ECG”) signal) can be monitored to provide information about a patient's cardiac health. One method of measuring a patient's ECG signal involves using external electrodes (e.g., 12 external electrodes or 5 external electrodes) that are attached to the patient's skin. External skin electrodes may be uncomfortable for the patient, and external leads attached to the external electrodes may be cumbersome or inconvenient for a patient to wear for long periods of time, or while performing their normal day-to-day activities. These and other challenges with external monitoring systems can lead to poor patient compliance, poor data collection, and a solution unsatisfactory for monitoring for long periods of time (e.g., weeks, months, years).

Implantable loop recorders have also been used to record ECG signals for patients, and typically use two electrodes on the implantable loop recorder to sense the ECG signal. Implantable loop recorders are typically battery powered and are implanted subcutaneously in the patient's chest region. After storing the recorded ECG signal information to internal memory, such as static random access memory (“SRAM”) of the implantable loop recorder, and before the SRAM has been filled with data to avoid overwriting data and loss of data, the implantable loop recorder can wirelessly transmit the data by radio frequency (“RF”) telemetry to an external device.

Power consumption in implantable loop recorders is a concern, because it is inconvenient to replace the battery of the recorder or to implant a new recorder. Frequent recording and storing of ECG signals can increase the frequency with which data must be telemetered from the device, or risk losing the data, and the RF telemetry burden on the implantable loop recorder can significantly reduce battery life for the implantable loop recorder.

Implantable loop recorders typically store and transmit detected episodes of cardiac arrhythmic events based on embedded detection algorithms. Due to limitations on power consumption, implantable loop recorders typically store and transmit only short durations (for example, a total of 60 minutes) of ECG signal data.

### BRIEF DESCRIPTION OF THE DRAWINGS

FIG. 1 is a diagram of an example system configured to communicate using a Galvanic communication link, in accordance with one or more implementations.

FIG. 2 is a block diagram of an example system configured to communicate using a Galvanic communication link, in accordance with one or more implementations.

FIG. 3 is a block diagram of an example transceiver that can be used for bidirectional communication of data over a Galvanic communications link, in accordance with one or more implementations.

FIG. 4A is a diagram of an example external device and an example implantable medical device with a first example electrode configuration, in accordance with one or more implementations.

FIG. 4B is a diagram of an example external device and an example implantable medical device with a second example electrode configuration, in accordance with one or more implementations.

FIG. 5 is a block diagram of an example implantable medical device, in accordance with one or more implementations.

FIG. 6 is a flowchart of example operations that an implantable medical device can perform, in accordance with one or more implementations.

FIG. 7A is a bottom view of an example external device, illustrating a first example electrode configuration, in accordance with one or more implementations.

FIG. 7B is a bottom view of another example external device, illustrating a second example electrode configuration, in accordance with one or more implementations.

FIG. 7C is a bottom view of yet another example external device, illustrating a third example electrode configuration, in accordance with one or more implementations.

FIG. 8A is a side view of an example implantable medical device implanted at a subcutaneous location in a thorax of a patient, and an example external device positioned over the implantable medical device, in accordance with one or more implementations.

FIG. 8B is a front view of an example implantable medical device implanted at a subcutaneous location in a thorax of a patient, and an example external device positioned over the implantable medical device, where the external device includes example auxiliary ECG or EEG measurement electrodes, in accordance with one or more implementations.

FIG. 9 is a block diagram of an example device that may be used external of a patient's body, and that may communicate with an implantable medical device, in accordance with one or more implementations.

FIG. 10 is a system diagram of an environment that includes an implantable medical device, an external device, a smartphone, a base station, cloud connected computing devices, cloud storage, and various Internet of Things (IOT) devices, in accordance with one or more implementations.

FIG. 11 shows an example operation of a simple key encryption system, in accordance with one or more implementations.

FIG. 12 shows an example operation of a public key encryption system, in accordance with one or more implementations.

FIG. 13 shows an example data compression operation using a Huffman algorithm, in accordance with one or more implementations.

FIG. 14 shows an example operation of an encoder using a forward error correction code, in accordance with one or more implementations.

FIG. 15 shows an example operation of a decoder using a forward error correction code, in accordance with one or more implementations.

FIG. 16 shows the structure of the communications layers that are implemented by the invention.

Like reference symbols in the various drawings indicate like elements.

### DETAILED DESCRIPTION

Described herein are devices, systems and methods that can be used to efficiently monitor, record, and analyze one or more physiological signals, such as one or more of electrical signals from the heart (e.g., ECG signal), a cardiac auditory or mechanical signal, or other physiological signals such as EEG. Some implementations of the devices, systems and methods described herein include an implantable medical device and a device external of the body of a patient. In some implementations, the implantable medical device and the external device may communicate using galvanic communications, such as by establishing a galvanic communications link between the implantable device and the external device, where the body tissue of the patient between the devices serves as the conductive medium over which the communications signals travel or are conducted. In this manner, the implantable medical device may transfer digital data to the external device (or may receive digital data from the external device), where the transmission medium is patient body tissue, and without relying on signal transmission through air outside of the patient's body. In some implementations, the implantable medical device and the external device may communicate without using an RF antenna, such as without using an RF antenna at the implantable device and without using an RF antenna at the external device. In some implementations, the implantable medical device and the external device may communicate without using a carrier signal, for example without modulating at the transmitting end data onto a carrier wave or signal or demodulating data from a carrier at the receiving end. In some implementations, the implantable medical device may communicate with the external device by transmitting, receiving, or both transmitting and receiving, data via the same electrodes that the implantable device uses to sense one or more physiological signals. In some implementations, the implantable medical device may communicate with the external device by transmitting, receiving, or both transmitting and receiving, data using at least one of the electrodes that the implantable device uses to sense one or more physiological signals (e.g., by using one of the same electrodes used to sense, and by using another electrode).

In some implementations, the implantable medical device is an implantable cardiac signal-monitoring device configured to be subcutaneously implanted in a chest region of a patient, but other types of implantable medical devices (e.g., cardiac pacemaker, cardioverter-defibrillator, neurostimulator, left ventricular assist device, artificial heart valve, coronary stent, insulin pump, devices to treat incontinence, impotence, or obesity, orthopedic implants, such as a bone plate that can monitor, e.g., share of the force/patient weight carried, or other type of implantable medical devices) may alternatively be used. In some implementations, a physiological signal is continuously monitored. In some implementations, a physiological signal is continuously recorded. In some implementations, the physiological signal is continuously analyzed. In some implementations, the physiological signal is continuously monitored and recorded. In some implementations, the physiological signal is continuously monitored, recorded, and analyzed.

For example, with some implementations of the devices, systems, and techniques discussed herein, a patient's physiological data signal (e.g., ECG and EEG, or other physiological signal, or multiple physiological signals) may be continuously recorded and stored within an implantable medical device (e.g., continuously recorded over a period of at least 4 hours, at least 8 hours, at least 12 hours, at least 24 hours, at least 2 days, at least 4 days, at least 1 week, at least 2 weeks, at least 1 month, at least 2 months, at least 6 months, at least 1 year, or longer). The data representing the continuously recorded signal may be periodically transmitted from the implantable medical device to a device external of the patient's body, via a low-power, high-speed communications technique that preserves power of the implantable device. The frequency of upload from the implantable device to the device external of the patient's body any appropriate frequency, such as, in various implementations, daily (or multiple times per day in some examples), every couple of days, weekly, biweekly, monthly, or yearly, or other appropriate frequency. In various implementations, the data received at the external device may be analyzed at the external device, including using one or more machine-learning algorithms to process and analyze the data, or using one or more artificial intelligence algorithms to process and analyze the data. In some implementations, the external device may transmit the received data to a cloud-based storage location, and the data may be analyzed by another computing device, such as a super-computer with access to the cloud, including using one or more machine-learning algorithms to process and analyze the data, or using one or more artificial intelligence algorithms to process and analyze the data. Results of the analyses can be used to modify or optimize parameters of the implantable medical device in some implementations, to alert the patient of a predicted medical event, or to alert a physician or clinician of recommended changes to the patient's medication, or to dosages of the patient's medication. This may benefit patients with management of syncope or atrial fibrillation or other cardiac arrhythmias, and may be predictive to help avoid a cardiac event such as ischemia, myocardial infarction, acute heart failure, or stroke. Because of the low power burden on the implantable medical device, such monitoring may be provided over long periods of time, such as 3 years or more, 5 years or more, or 10 years or more, without needing to replace a battery of the implantable medical device, according to some implementations.

In some implementations, the implantable medical device records a physiological signal based on a detected physiological event. In some implementations, the implantable medical device periodically records a physiological signal, or based on timing event (e.g., expiration of a timer). In some implementations, the implantable medical device records a physiological signal based on patient-provided input. In some implementations, the implantable medical device records a physiological signal based on an input received from another device (e.g., a communication received from another device). In some implementations, the implantable medical device records a physiological signal by storing data associated with the physiological signal in memory of the implantable medical device (e.g., SRAM, flash memory, dynamic random access memory (DRAM), or other type of memory). In some implementations, the implantable medical device initially stores data associated with the physiological signal in SRAM, and then later moves the data from SRAM to flash memory. In some examples, the data is filtered, compressed, or filtered and compressed before storage to memory. [00030] In some implementations, the device external of the body of the patient is configured to communicate with the implantable device using one or more communication methods, such as one or more of galvanic communication, conducted communication, RF communication (e.g., Medical Implant Communications System (“MICS”), Bluetooth Low Energy (“BLE”)), or other type of communication. In some examples, the external device includes three or more (e.g., four, five, six, seven, eight, nine, ten, eleven, twelve, or more) electrodes, and determines which two of the three or more electrodes to use in communicating with the implantable medical device via a Galvanic communications link. For example, the external device may determine which two of the three or more electrodes may be appropriately aligned, as will be described further below, with the electrodes of the implantable medical device, so that signals transmitted between the devices may be received with minimal attenuation or reduced attenuation. In some examples, the external device includes two electrodes that it uses to communicate via a galvanic communications link with the implantable medical device.

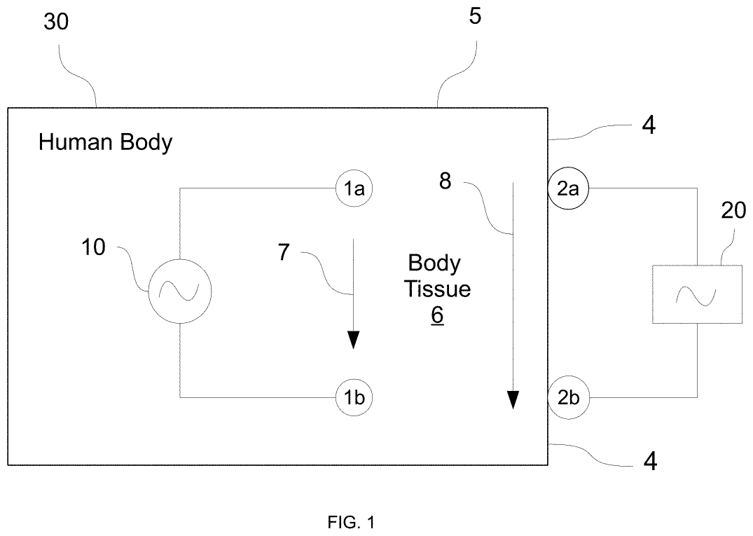

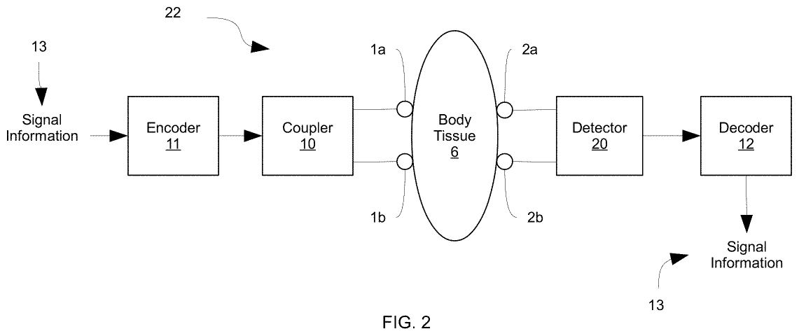

FIG. 1 is a diagram of an example system 5 configured to communicate using a Galvanic communication link, in accordance with one or more implementations. The system 5 includes a coupler 10 and a pair of electrodes 1a, 1b that are implanted within a human body 30, and a detector 20 and a pair of electrodes 2a, 2b that are external of the human body 30, with the external electrodes 2a and 2b in contact with skin 4 of the patient. In some examples, the Galvanic communication link may provide a physical communication layer for communications between an implanted device and a device external of a patient's body. In some examples, the communication link may be unidirectional (e.g., from implanted device to external device, or from external device to implanted device), and in some examples the communication link may be bidirectional. Coupler 10 is a signal source that may create square wave signals, where the signals can represent digital data (e.g., 1's and 0's), such as data stored in an implantable medical device. The square wave signals flow through body tissue 6 of the human body 30, creating a primary current 7 near the implanted electrodes 1a, 1b. The primary current 7 creates an electric field that propagates through the body tissue 6 between the implanted electrodes 1a, 1b and the external electrodes 2a, 2b, and causes a secondary current 8 at the skin surface 4 between external electrodes 2a and 2b, where the secondary current 8 can be detected by detector 20 external of the human body 30. The system 5 depicted in FIG. 1 shows an example of basic Galvanic coupling. The system 22 of FIG. 2 will expand on the system 5 of FIG. 1 to show additional components that can be used in a Galvanic communication channel between an implantable medical device and a device external of the patient's body, according to some implementations.

FIG. 2 is a block diagram of an example system 22 configured to communicate using a Galvanic communication link, in accordance with one or more implementations. The system 22 includes the coupler 10 and detector 20 of FIG. 1, and adds an encoder 11 and a decoder 12. The encoder 11 can add data layer encoding to a stream of data 13 (e.g., physiological signal information, other data) received at the encoder 11, and then pass the encoded data to the coupler 10. The data layer can implement the definition of digital data (e.g., a data ‘1’ or data ‘0’), and the physical layer can transmit through the body tissue 6. As described above with respect to FIG. 1, the coupler 10 can generate pulses, which can create a communication link conducted through the body tissue 6, and the detector 20 can detect a current at the skin electrodes 2a, 2b. Decoder 12 can remove the data layer encoding, recovering the stream of data 13 (e.g., physiological signal information, other data).

Various encoding and decoding techniques can be used. One example of an encoding and decoding technique that can be used is Manchester encoding. In some implementations, any encoding/decoding technique that uses a biphasic signal pattern may be used. Use of a biphasic signal pattern may provide neutrality in charge balance so that charge levels may not build up or accumulate in body tissue, which could cause tissue damage in some cases. In some implementations, the encoder 11 can ensure that the signal applied by the coupler 10 to the body tissue 6 is charge-balanced to a zero net DC charge. This can prevent accumulation of DC voltage in the body tissue 6 or elsewhere in the human body 30, which can be helpful in avoiding tissue damage and in avoiding disruption of decoder operations when communicating with other devices. Other types of encoding techniques, which may be used according to various implementations, include pulse position, pulse duration and pulse amplitude encoding.

In some implementations, a benefit communicating via a Galvanic communication link as composed to using RF telemetry, for example, is that the Galvanic communication link can be established without modulating data onto a carrier wave or demodulating data from a carrier wave, as occurs in RF telemetry systems. By comparison, RF telemetry systems may generally modulate data onto a high frequency wave (e.g., a high frequency sine wave) that is used as a transmission medium. The energy required to create this high frequency carrier waveform for RF telemetry is significant, and can cause undue power dissipation and deplete the battery of implantable devices that rely on RF telemetry to communicate data from the implant to external devices. Transmitting data via telemetry consumes significant power on a per bit basis, and the size of the battery or longevity of the device can be significantly (negatively) impacted by large data transfers. Because the implantable medical devices described herein may communicate with a device external of the body using a Galvanic communication link, without using a high frequency carrier wave and without the need for modulation/demodulation, substantial amounts of power may be saved in some implementations, which can extend battery life of the implantable medical device. By comparison, the power requirements for the Galvanic communications links between an implantable medical device and a device external of the patient's body may be much lower than traditional, RF-based telemetry communications methods between implantable medical devices and external devices.

Another advantage of the Galvanic communication, according to some implementations, is that the signal is pushed through the body tissue as ionic conduction, rather than being carried by electromagnetic (“EM”) waves. Since EM waves are more prone to noise and are absorbed by the tissue, data transmission via RF must be performed at slower rates as compared to Galvanic communications, and due to errors occurring in the data stream, communications more often need to be re-sent as compared to Galvanic communications, which in turn reduces the throughput rate for RF communications and increases the power consumed with RF communications.

Because power requirements for the Galvanic communications links discussed herein may be low, the implantable medical devices discussed herein may regularly transmit large amounts of data in comparison to traditional implantable medical devices, according to some implementations, and at much higher speeds, which may provide an additional benefit. For example, the implantable medical devices discussed herein may transfer data on the order of megabytes, whereas traditional implantable medical devices, limited by power-hungry communication techniques, may have transmitted data on the order of kilobytes, because the joules of energy required to transfer each bit of data using RF telemetry is so high compared to the energy required to transfer each bit of data using a Galvanic communications channel.

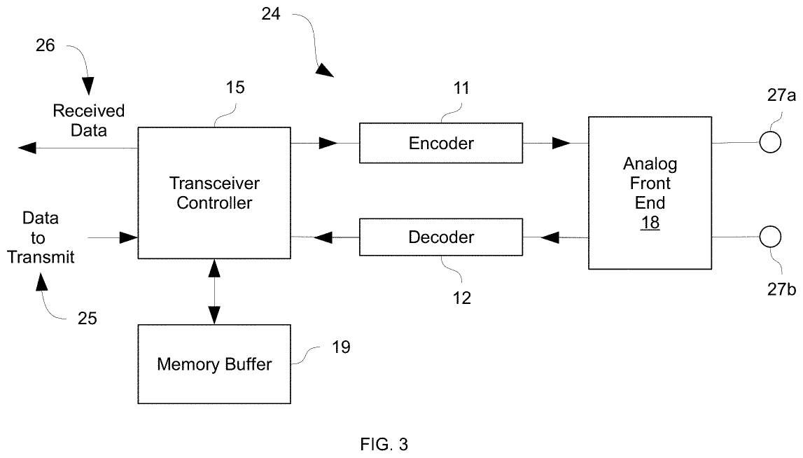

For simplicity, FIGS. 1 and 2 have generally depicted data transfer unidirectionally from an implantable medical device to a device external of a patient's body, but as discussed above data transfer may be bidirectional in some implementations. FIG. 3 is a block diagram of an example transceiver 24 that can be used for bidirectional communication of data over a Galvanic communications link, in accordance with one or more implementations. In some implementations, the transceiver 24 is included in an implantable medical device. In some implementations, the transceiver 24 is included in an external device. Transceiver 24 may transmit or receive data over a Galvanic communications link, according to various implementations.

In the transmit direction, data 25 to transmit can be provided to a transceiver controller 15, which can send the data 25 to the encoder 11. The encoder 11 can encode the data 25 and send the encoded data to an analog front end 18, which can convert the encoded data to waveforms and present, through electrodes 27a and 27b, the waveforms to a patient's body. For example, if transceiver 24 is included in an implantable medical device, electrodes 27a and 27b may be in contact with body tissue within a human body, and alternatively if transceiver 24 is included in an external device, electrodes 27a and 27b may be in contact with skin.

Data can be received by the transceiver 24 when the analog front end 18 receives physical layer waveforms at electrodes 27a and 27b and provides the received waveforms to the decoder 12. The decoder 12 can convert the received waveforms into digital data (e.g., 1's and 0's), and can interpret the digital data and decode the digital data. The decoder 12 can provide the decoded digital data to the transceiver controller 15, which can either send the received data 26 to a processor for further processing, or to a memory buffer 19, in various implementations. In various implementations, memory buffer 19 may be memory internal of the transceiver 24, or may be one or more memory units outside of the transceiver (e.g., SRAM, flash memory, other types of memory). For example, if transceiver 24 is included in an implantable medical device, electrodes 27a and 27b may be in contact with body tissue within a human body, and alternatively if transceiver 24 is included in an external device, electrodes 27a and 27b may be in contact with skin.

As described above, the implantable medical device may sense and record one or more physiological signals. For example, the implantable medical device may sense and record one or more of an ECG or EEG signals, a cardiac auditory signal, an indication of patient motion, an indication of patient respiration, an indication of patient temperature, an indication of patient pressure (e.g., blood pressure), cardiac acoustic or cardiac mechanical signals, an indication of tissue impedance, an indication of patient posture, or other physiological signals.

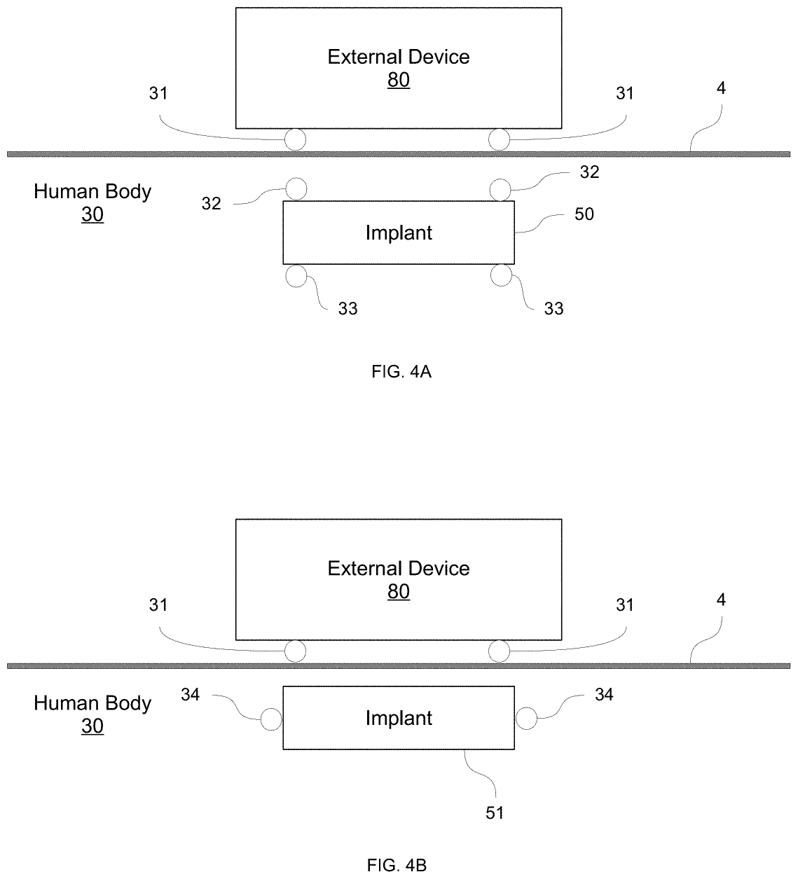

FIG. 4A is a diagram of an example external device 80 and an example implantable medical device 50 with a first example electrode configuration, in accordance with one or more implementations. External device 80 includes electrodes 31 that are in contact with a patient's skin 4. In some examples, external device 80 includes two electrodes 31, but in other examples external device 80 includes more than two (three, four, five, six, seven, eight, nine, ten, eleven, twelve, or more) electrodes. In some implementations, the external device 80 uses the electrodes 31 to communicate with the implantable medical device 50, for example by a Galvanic communication link.

Implantable medical device 50 includes four electrodes. Two electrodes 32 can be used for communicating with the external device 80, for example by a Galvanic communication link. As can be seen in FIG. 4A, the electrodes 32 may be oriented towards face the skin surface 4. In some implementations (not shown), implantable medical device 50 can include more than two electrodes 32 for use in communicating with the external device 80. Two electrodes 33 can be used to sense one or more physiological signals so that the one or more physiological signals can be recorded. The electrodes 33 may be oriented to face towards cardiac tissue, according to some implementations. Because the implantable medical device 50 may be subcutaneously implanted just beneath the patient's skin 4, the electrodes 33 are not in contact with the patient's heart, are not within a chamber of the patient's heart, and are not in contact with cardiac tissue. In some examples, the electrodes 33 may be spaced apart from one another an appropriate distance to better sense a desired ECG vector. Implant 50 includes four electrodes—two electrodes 32 for communication with an external device and two electrodes 33 for sensing or measuring one or more physiological signals, and the orientation of each of the electrodes 32, 33 may provide optimized functionality with regard to communicating with an external device and sensing and measuring physiological data. In some implementations, implantable medical device 50 may simultaneously communicate with external device 80 via communication electrodes 32 and sense and record physiological signal information via sense electrodes 33.

FIG. 4B is a diagram of an example external device 80 and an example implantable medical device 51 with a second example electrode configuration, in accordance with one or more implementations. External device 80 includes electrodes 31 that are in contact with a patient's skin 4, as described above with reference to FIG. 4A. Implantable medical device 51 includes two electrodes 34, which can be used both for communicating with the external device 80, for example by a Galvanic communication link, and for sensing one or more physiological signals so that the one or more physiological signals can be recorded. As can be seen in FIG. 4B, the electrodes 34 may be located generally at distal longitudinal ends of the implantable medical device 51, although other locations are possible as well. Because the implantable medical device 51 may be subcutaneously implanted just beneath the patient's skin 4, the electrodes 34 are not in contact with the patient's heart, are not within a chamber of the patient's heart, and are not in contact with cardiac tissue. In some implementations (not shown), implantable medical device 51 can include more than two electrodes 34 that can be used both for communication and physiological signal sensing.

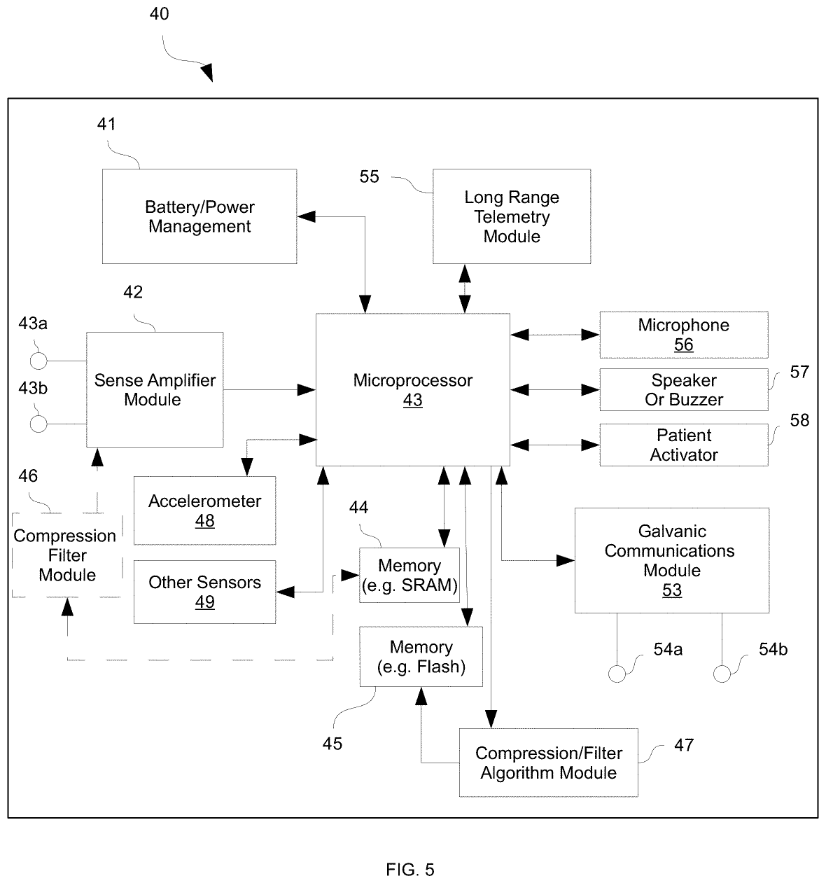

FIG. 5 is a block diagram of an example implantable medical device 40, in accordance with one or more implementations. The implantable medical device includes a battery 41, may also include power management circuitry in some implementations. In some implementations, battery 41 may be one or more primary cell battery. In some implementations, battery 41 may be one or more rechargeable battery. In some implementations, device 40 includes one or more primary cell batteries and one or more rechargeable batteries. Implantable device 40 may operate for a period of at least three years, and up to ten years, or longer, on a primary cell battery, according to some implementations. For implementations that include a rechargeable battery, the rechargeable battery may be recharged by an external device, for example. Longevity of the implantable device 40 may be important for implementations where the implantable device 40 is expected to be used for monitoring periods of multiple years, or for chronic disease management for multiple years.

The implantable medical device 40 also includes a sense amplifier module 42 that includes one or more sense amplifiers, which can be used to sense one or more physiological signals via electrodes 43a, 43b that may reside on an external surface of the implantable medical device 40 and may contact body tissue when the device 40 is implanted. One example of a physiological signal that the sense amplifier module 42 can sense is an ECG signal. The electrodes 43a, 43b can be orientated and separated to pick up desired ECG signals from the patient's heart. In some examples, the electrodes 43a, 43b may be platinum, platinum iridium (90/10), or stainless steel, and may be biocompatible and non-corrosive. In various implementations, signals sensed by the one or more sense amplifiers can be converted to a digital signal either by the sense amplifier module 42, or by a processor 43 having an on-board or off-chip analog to-digital converter.

In some implementations, the sense amplifier module 42 and electrodes 43a, 43b can measure tissue impedance. Tissue impedance can be used to measure a surrogate of body wetness, for example, and thereby be used to detect fluid retention or weight gain. Impedance trend over time may provide useful information independently, or in combination with other data such as ECG data or accelerometer data that may indicate patient activity. Similarly, the electrodes 43a, 43b may measure impedance of the lungs which may indicate fluid buildup or changes in the lungs over time. In some examples, fluid in the lungs may indicate a heart failure condition. Detection of these changes early can allow for intervention, such as medication changes or clinical interventions, which may prevent patient hospitalization or death.

The processor 43 can execute instructions that can cause various actions to occur throughout the implantable medical device 40. For example, the processor 43 can process sensed signal data from the sense amplifier module 42, and can filter the signal, compress the data, and execute detection algorithms on the data, according to various implementations. Detection algorithms may be used to detect, without limitation, heart rate, arrhythmias, atrial fibrillation, ST segment elevation changes (e.g., elevation changes in the ST-segment of the ECG), electrical impedance, and posture. In some examples, the processor 43 may use template matching to compare a sensed ECG signal with various ECG signatures. In some implementations, the implantable medical device 40 may receive information that can be used with the one or more detection algorithms from one or more external devices, including remote computing devices, cloud-connected devices, smartphones, external programmers, and the like. Information that can be received can include, without limitation, template information, threshold information, waveform characteristics that are known or expected to indicate current or future cardiac conditions or states, such as a future syncope event, tachycardia, bradycardia, atrial fibrillation, myocardial infarction, or acute heart failure, to list just a few examples.

The processor 43 can store data to (or read data from) memory of the implantable medical device 40. The implantable medical device 40 can include various types of memory. Shown in FIG. 5 is static memory 44 (e.g., SRAM), and nonvolatile memory 45 (e.g., flash memory). In some examples, the processor 43 may initially store captured data (e.g., ECG signal data or information associated with the ECG signal data) to static memory 44. SRAM memory may have low power requirements, but may be a volatile memory. When the static memory 44 has been filled to capacity (or some pre-capacity threshold), the processor 43 may transfer a block of data from the static memory 44 to the non-volatile memory 45 (e.g., flash memory). In some implementations, the implantable device 40 includes at least 5 megabytes of flash memory. In some implementations, the implantable device 40 includes at least 10 megabytes of flash memory. In some implementations, the implantable device 40 includes more than 10 megabytes of flash memory (e.g., at least 20 megabytes, at least 50 megabytes, at least 100 megabytes, at least 500 megabytes, at least one gigabyte, at least two gigabytes, five gigabytes, ten gigabytes, or more).

In some implementations, the processor 43 may enter a “sleep” mode during periods of inactivity to conserve power. An interrupt from the sense amp module 42 (or by a low power counter/timer (not shown)) may interrupt and wake up the processor 43 and cause the processor 43 to perform the static memory to nonvolatile memory data transfer. Some implementations include a first optional compression/filter module 46, and in some implementations the sense amplifier module 42 may store data directly to memory, or to memory through the compression filter module 46, where the data may be filtered, compressed, or filtered and compressed, according to various implementations. This may reduce the burden on the processor, which may result in power savings in some implementations. In some implementations, the processor 43 may batch-process data stored in static memory 44 for use in various algorithms. Compression of data can reduce an amount of data that is stored in the static memory 44, which will also reduce the amount of data subsequently stored to the non-volatile memory 45. This can save power associated with processor reads and writes of memory, for example, and can reduce the power needed to transfer the data to a device external of the body. In some examples, the functions of the processor 43 may also be implemented via hardware state machines, which may further reduce power consumption.

A compression/filter/algorithm module 47 can filter raw or less processed data, according to some implementations. FIG. 5 shows the compression/filter/algorithm module 47 as a standalone module for simplicity, but in some implementations the module 47 may be included within the processor 43. In some examples, the module 47 performs one or more of filtering, compression, and one or more algorithms on data presented to the module 47. The data may be passed to the module 47 by the processor 43 as it is received from the sense amplifier module 42 in some implementations. In some implementations, the processor 43 may read data from static memory 44 and pass the data to the compression/filter/algorithm module 47 for processing, such as for batch processing at specific time intervals. After processing the data, the compression/filter/algorithm module 47 may store the processed data to non-volatile memory 45, according to some implementations (or to static memory 44 in some implementations).

The implantable medical device 40, via the processor 43 or the compression/filter/algorithm module 47 in various implementations, can filter blocks of data or utilize algorithms to examine blocks of data to look back in time to interpret the data. By batch processing in this manner, power consumption may also be reduced as the processor or algorithm module 47 may only need to turn on intermittently to process the batch. The data may include, without limitation, the ECG waveform(s), accelerometer data, activity data, patient input data (voice, taps), piezo sensors, temperature data, pressure data, and the like. In some implementations, the processor 43 or compression/filter/algorithm module 47 can filter the data to remove unwanted noise such as electromagnetic interference, motion artifact, or white noise. In some implementations, the processor 43 or compression/filter/algorithm module 47 can compress the data to reduce the data file size that will be stored in nonvolatile memory 45 (e.g., flash) and subsequently transmitted outside the body. In some implementations, the processor 43 or compression/filter/algorithm module 47 can execute local algorithms (e.g., within the implantable medical device 40) that may detect cardiac events such as arrhythmias, atrial fibrillation, ST segment elevations, or template matching of ECG signals that can indicate a cardiac condition or future condition.

The compression/filter/algorithm module 47 may be enabled, disabled, or configured, for example, by a physician or by a cloud-computing device (or other device) based on the needs of the patient. For example, a local algorithm to detect atrial fibrillation could be turned on if the patient is known or expected to have frequent atrial fibrillation. The local algorithm may provide real-time notifications to the patient that atrial fibrillation is occurring, for example, and may instruct the patient to take medication or change medication when this occurs, according to some implementations. For patients that do not require real-time notifications, for example, such algorithms may be turned off so save battery power. Such patients may instead have their data analyzed via remote processing, such as by a device external of the patient's body following upload of the patient's data from the implantable medical device 40 to an external device, where the analysis may occur at the device to which the data was immediately uploaded, or at another external device, for example.

The implantable medical device includes an accelerometer 48, which can be used to monitor and record one or more of patient position or orientation, patient activity level, patient sleep cycle information, patient movement information, or the like. Such data can be used to interpret sensed ECG waveforms, based on the additional information data regarding patient activity. The accelerometer data may be helpful for diagnosing and trending a patient's health condition over time, and may also be helpful in optimizing the interpretation of ECG signals based on body position or motion artifact. In some implementations, one or more other sensors 49 (e.g., one or more temperature sensors, pressure sensors, piezo sensors, or other types of sensors) are included in the implantable medical device 40.

The implantable medical device includes a Galvanic communication module 53 that it can use to communicate via a Galvanic communication link with another device, such as a device external of the body of the patient or another implanted device. For example, the implantable medical device 40 can use the Galvanic communication module 53 to upload stored physiological data to a device external of the body. Examples of physiological data that can be uploaded to the external device can include, without limitation, ECG signal data, heart sound data, data from the accelerometer 48 (e.g., activity data, posture data, or other of the types of data described above with respect to the accelerometer), data from the one or more other sensors 49, patient-provided input data (e.g., voice commands, input taps), and the like. The processor 43 can utilize the Galvanic communications module 53 for very low power and high data rate transfers. The Galvanic electrodes 54a, 54b can be located on an exterior surface of the implantable medical device 40, can be of the same or similar materials as electrodes 43a and 43b, and can contact the patient body tissue at the implant location. In some implementations, the Galvanic electrodes 54a, 54b may be common with the sense electrodes 43a, 43b, as described above with reference to FIG. 4B. In implementations where the sense electrodes 43a, 43b and the communication electrodes 54a, 54b are separate, the sense electrodes 43a, 43b can be optimized (e.g., placement/orientation on device, spacing) for ECG detection, and the communications electrodes 54a, 54b can be optimized (e.g., placement/orientation on device, spacing) for communication with a device external of the body.

In some implementations, the implantable medical device 40 can include an electrical impedance measurement circuit that can be used to detect changes in the magnitude, phase, or magnitude and phase of an electrical impedance, which can be used to sense changes in the environment surrounding the implantable medical device 40. Such changes in the environment could be indicative of, for example, patient respiration. Such changes could also be indicative of, for example, fluid retention by the patient, or of local inflammation (or other conditions), and may provide information regarding a condition's (or a parameter's) manifestation over time, and in morphology.

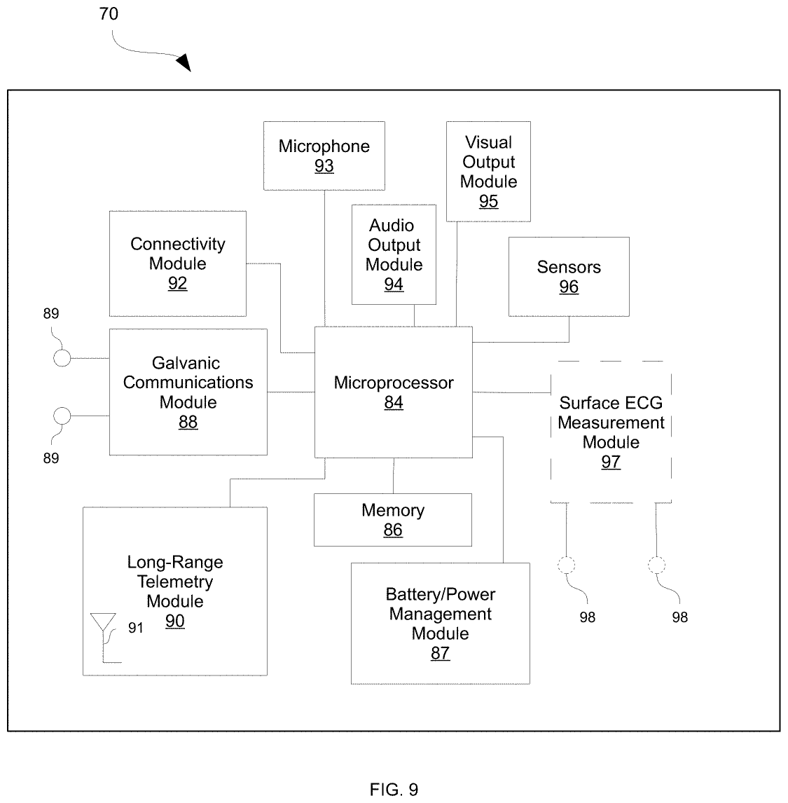

The implantable medical device 40 includes a long-range telemetry module 55 that can be used to communicate with devices external of the body of the patient over a distance of centimeters or meters. For example, the implantable medical device 40 may communicate with a smartphone, with a wearable smart device, with a base station, or with another computing device using the long-range telemetry module. In some implementations, the long-range telemetry module 55 may communicate using RF telemetry, such by using MICS, BLE or other RF communications protocol. In some implementations, implantable medical device 40 does not include the long-range telemetry module 55, and all communications with devices external of the patient's body may occur via the Galvanic communications module 53. In some examples, the implantable medical device 40 may detect an abnormality in a recorded physiological signal, and may transmit via RF telemetry to a device external of the body of the patient (e.g., smartphone 102, external device 70 base station 104, see FIG. 9) data associated with the physiological signal and the abnormality.

The implantable medical device 40 includes a microphone 56 that may be voice-activated, to detect speech or other audible sounds from the patient and allow for voice commands, according to some implementations. In some implementations, the microphone 56 may be used to detect obstructive sleep apnea via one or more of breathing sounds, heart valve sounds, and other respiratory sounds such as cough, wheezing, labored breathing, pneumonia, and the like. In some implementations, implantable medical device 40 does not include the microphone 56.

The implantable medical device 40 includes a mechanical buzzer or speaker 57, which can be used by the implantable medical device 40 to provide feedback to the patient. The feedback may be, for example, an indication that atrial fibrillation or other type of arrhythmia has been detected. As another example, the feedback may indicate that the patient is due to upload data to an external device. In some implementations, implantable medical device 40 does not include the buzzer of speaker 57.

The implantable medical device 40 includes a patient activator 58 that may allow the patient to tap the implantable medical device 40 to mark data currently being recorded or data that was previously recorded over a predetermined time period or data that will be recorded in the future over a predetermined time period. For example, if the patient does not feel well, has a fainting spell, or the like, the patient may apply a specific tap to the implantable medical device 40, which can be detected by the patient activator 58, to place a marker on the data.

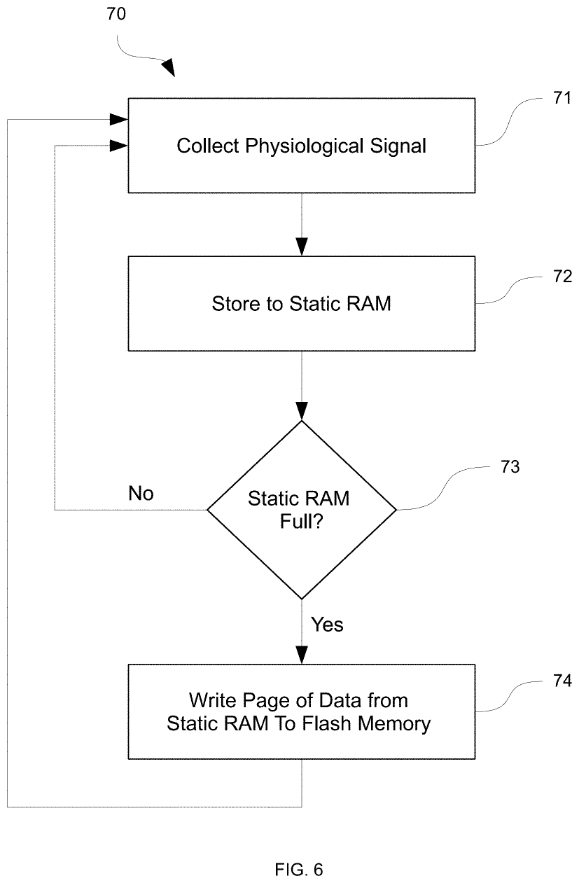

FIG. 6 is a flowchart 70 of example operations that an implantable medical device can perform, in accordance with one or more implementations. At a first step 71, the implantable medical device can collect a physiological signal, such as any of the types of physiological signals discussed herein. The implantable medical device can store the physiological signal, or data or information associated with the physiological signal, to static RAM at step 72. At step 73, if the static RAM is not full, the implantable medical device can collect a physiological signal at step 71 at an appropriate time. If the static RAM is full at step 73, the implantable medical device can write a page of data from the static RAM to flash memory at step 74. After the write to flash memory, the implantable medical device can collect a physiological signal at step 71 at an appropriate time. In general, writes to static memory may require less power than writes to flash memory.

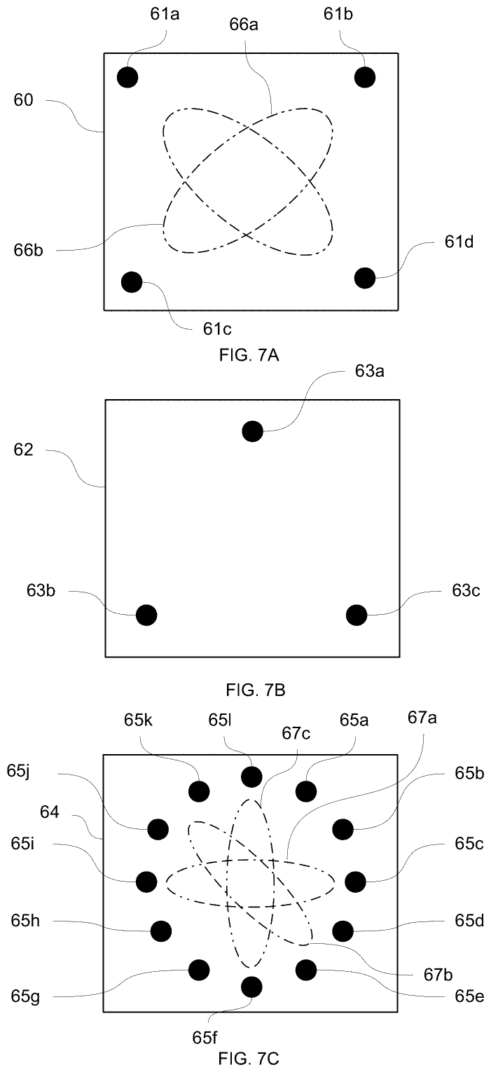

As described above, in some implementations, the device external of the patient's body can include more than two communications electrodes for use in communicating with the implantable medical device over a Galvanic communication link. FIG. 7A is a bottom view of an example external device 60, illustrating a first example electrode configuration, in accordance with one or more implementations. Device 60 includes four electrodes 61a, 61b, 61c, 61d, arranged generally in a square pattern. FIG. 7B is a bottom view of another example external device 62, illustrating a second example electrode configuration, in accordance with one or more implementations. Device 62 includes three electrodes 63a, 63b, 63c, arranged generally in a triangle pattern. FIG. 7C is a bottom view of yet another example external device 64, illustrating a third example electrode configuration, in accordance with one or more implementations. Device 64 includes twelve electrodes 65a, 65b, 65c, 65d, 65e, 65f, 65g, 65h, 65i, 65j, 65k, 65l, arranged generally in a circular pattern. By providing more than two electrodes with each of the configurations shown in FIGS. 7A-7C, communications over a Galvanic communication link may occur generally using any two of the electrodes of the respective external device. The two external device communications electrodes that are the most near-parallel and the most closely aligned to the communications electrodes of the implanted device may provide the best signal reception, and by communicating over those electrodes may advantageously permit the lowest Galvanic power output from the implanted device.

For any radial orientation of the external device 60, 62, or 64 when the patient positions the external device 60, 62, or 64 against her skin over the implanted medical device, two of the electrodes in the respective set of electrodes 61, 63, 65 may be near-parallel with the electrodes of the implantable medical device, according to some implementations. In some implementations, Galvanic communications with the implant may be improved using the two electrodes that are near-parallel with the implant communication electrodes, and the external device 60, 62, or 64 may determine which two electrodes from the respective set of electrodes 61, 63, 65 to use for communicating with the implantable device.

For example, circuitry and/or software within the external device 60, 62, or 64 can monitor received Galvanic communications signals from the implantable device over various pairs of electrodes on the external device 60, 62, or 64, and select a pair of electrodes over which the signal strength of the received communications is strongest for a given orientation of the external device 60, 62, or 64. In some examples, this determination and selection can be done once at the beginning of the communications session.

In some examples, the determination and selection may be done multiple times (e.g., periodically, semi-continuously) during the communications session to accommodate for movement of the external device 60, 62, or 64, and in some cases the optimal pair of electrodes may change during the communications session. That is, during a first portion of a Galvanic communications session with the implantable device, the external device 60, 62, or 64 may communicate using a first pair of electrodes, and during a second portion of the communications session the external device 60, 62, or 64 may communicate using a second pair of electrodes, where at least one of the electrodes in the second pair is different from the electrodes of the first pair. In some examples, selecting the best electrode pair at the external device 60, 62, or 64 can reduce the power needed to establish and maintain the Galvanic communications link, which extends battery life and reduces the size of the device. Such reduced power benefits may apply at the implantable device, at the external device 60, 62, or 64, or at both devices, according to some implementations. While three patterns of electrodes were shown in FIGS. 7A-7C, any appropriate pattern of electrodes can be used, with any appropriate number of electrodes (two, three, four, five, six, seven, eight, nine, ten, eleven, twelve, or more electrodes).

FIG. 7A shows two possible orientations 66a, 66b of an implantable medical device, or to state another way—orientations of the external device 60 for a given implanted location of the implantable medical device. With orientation 66a, assuming the implantable device has communication electrodes positioned near the distal, longitudinal ends of the implantable device, the external device 60 may select the communication electrodes 61b and 61c for communication with the implanted device. Similarly, for orientation 66b, the external device 60 may select the communication electrodes 61a and 61d for communication with the implanted device.

FIG. 7C shows three possible orientations 67a, 67b, 67c of an implantable medical device, or to state another way—orientations of the external device 64 for a given implanted location of the implantable medical device. With orientation 67a, assuming the implantable device has communication electrodes positioned near the distal, longitudinal ends of the implantable device, the external device 64 may select the communication electrodes 65c and 65i for communication with the implanted device. Similarly, for orientation 67b, the external device 64 may select the communication electrodes 65e and 65k, and for orientation 67c the external device 64 may select the communication electrodes 65f and 65l for communication with the implanted device.

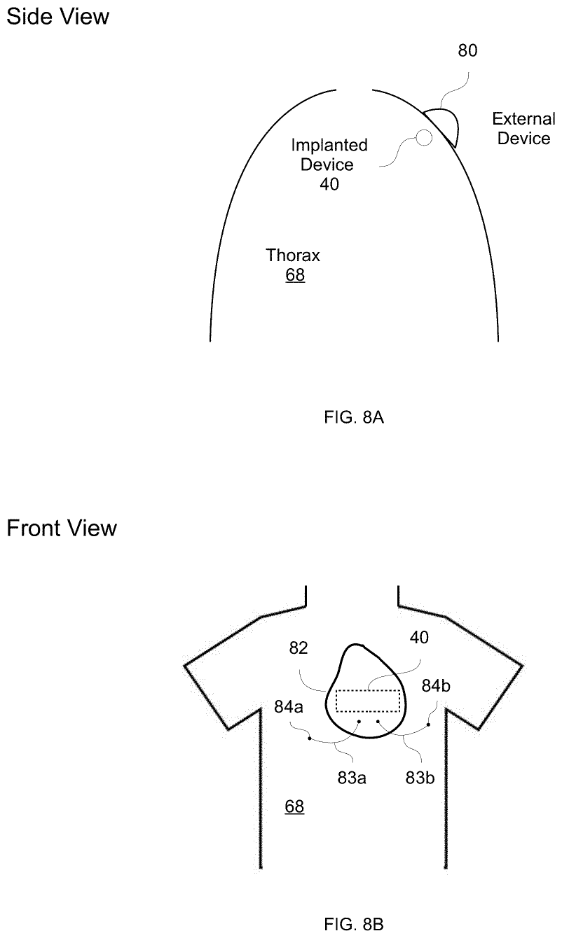

FIG. 8A is a side view of an example implantable medical device 40 implanted at a subcutaneous location in a thorax 68 of a patient, and an example external device 80 positioned over the implantable medical device 40, in accordance with one or more implementations. As can be seen in FIG. 8A, the external device 80 can be positioned proximal the subcutaneously implanted device 40, where the external device 80 may be positioned by the patient on the patient's skin over the implanted device. The external device 80 can be held in position by the patient so that communications between the devices 40, 80 can take place, and the patient can, for example, coarsely locate the implanted device 40 and hold the external device 80 over the location of the implanted device 40.

FIG. 8B is a front view of an example implantable medical device 40 implanted at a subcutaneous location in a thorax 68 of a patient, and an example external device 82 positioned over the implantable medical device 40, where the external device 82 includes example auxiliary ECG measurement electrodes 84a, 84b, in accordance with one or more implementations. External device 82 includes auxiliary ECG electrodes 84a, 84b that can be used to measure a patient's ECG signal, and compared against the measured ECG signal from the implanted device 40, for example. Adjustments can be made to sense algorithms or measurement algorithms of the implanted device based on the comparison, according to some implementations. Electrodes 84a, 84b may be ECG patches in some examples, and may be electrically connected to the external devices by wires 83a and 83b, respectively. In some examples, wires 83a, 83b may be permanently attached to the external device 82, and in some examples the wires 83a, 83b may be detachable from the external device 82. In some examples, the ECG electrodes 84a, 84b may be connected to spring-loaded spools that may allow the wire to be pulled out, and then retracted back.

FIG. 9 is a block diagram of an example device 70 that may be used external of a patient's body, and that may communicate with an implantable medical device, in accordance with one or more implementations. In some examples, device 70 may correspond the external device 80 of FIGS. 4A and 4B. Device 70 may be used to receive data (e.g., physiological signal data) from an implantable medical device, where the data may be communicated from the implantable medical device to the external device 70 over a Galvanic communication link, and device 70 may then either analyze the received data locally at device 70 or may transmit the data received from the implantable medical device to another device external of the patient, such as a cloud connected device.

The external device 70 includes a processor 84, which may include processing power sufficient to provide machine-learning algorithms, artificial intelligence algorithms, or both machine-learning (“ML”) algorithms and artificial intelligence (“AI”) algorithms to process data received from the implantable medical device (i.e., such that those algorithms may run locally on the external device 70). For example, the external device can include ML and AI algorithms implemented locally in the processor 84. The ML or AI algorithms may, in some implementations, learn patient-specific characteristics over time, and may use the learned characteristics to optimize detection and prediction of events. In some implementations, the ML or AI algorithms may use patient activity data (eating activity, sleeping activity, exercise activity, and the like) and related information (e.g., time of day that the activity occurs) to optimize detection and prediction of events. In some implementations, the ML or AI algorithms may use patient input of occurrence of clinical events (for example, syncope, myocardial infarction, cardiac arrhythmias, stroke, or acute heart failure) to optimize detection and prediction of events. In some examples, the local ML and AI algorithms at the external device 70 may be revised or updated from time to time based on ML or AI algorithms performed on one or more other computing devices, such as cloud connected computing devices, where the one or more other computing devices may provide the revision or update to the external device 70.

The external device includes memory 86 (SRAM, flash, DRAM, or other types of memory) where data and instructions can be stored, for example. The external device 70 includes a battery/power management module 87, which includes one or more batteries and circuitry to monitor power management for the external device 70. In some examples the power management module can provide a battery status to the user. The one or more batteries may be rechargeable, and in various implementations may be recharged by a base station, by a power cord, or by other recharging techniques. In implementations where the implantable medical device includes a rechargeable battery, the battery/power management module 87 may be used to recharge the battery of the implantable medical device, according to some implementations.

The external device 70 includes a Galvanic communications module 88 can be used to communicate via a Galvanic communication link with another device, such as an implanted medical device. For example, physiological signal data stored at the implanted medical device may be uploaded via a Galvanic communication link to the external device 70, where the data may be analyzed at the external device 70, or in some cases transmitted to another device, such as a cloud-connected device, for analysis. In some examples, the external device can use the Galvanic communications module 88 to send commands, markers, updates, or revisions to the implant. The Galvanic communications module 88 can include a transceiver (e.g., a transmitter and a receiver) capable of Galvanic communications, and two or more communications electrodes 89, which may be on a bottom outside surface of the external device for contact with the skin of the patient. As described above with reference to FIGS. 7A-7C, more than two communications electrodes 89 are possible.

The external device 70 includes a long-range telemetry module 90 with an antenna 91 (e.g., an RF antenna) that can be used to communicate with other devices. The long-range telemetry module 88 includes one or more transceivers that can communicate using one or more RF telemetry communications protocols, such as BLE or MICS. For implementations of the implanted medical device that include a long-range telemetry module at the implantable device, the external device 70 and the implantable device may communicate via RF telemetry (e.g., via BLE or MICS). In various implementations, the external device 70 may also communicate with other devices, such as a smartphone, a wearable smart device, a base station, or other devices via the long-range telemetry module 88. Long-range telemetry communications can be useful for sending smaller data files and alerts that can be received real-time, rather than waiting for a Galvanic data exchange, which may be scheduled to occur at predetermined periods (e.g., daily, weekly, monthly, or another predetermined period). Alerts may be sent to the patient or to the physician, according to some implementations.

A connectivity module 92 may be used to communicably connect the external device 70 to one or more other devices via WiFi, wired internet, mobile data, or other connectivity techniques. For example, the external device 70 may communicate or communicably connect with one or more cloud-connected devices using the connectivity module. In some implementations, the external mobile device may upload to a cloud-storage location data previously uploaded from the implantable medical device to the external device 70. In some examples, the ML or AI algorithms executing locally at the external device 70 to process patient data can provide data or results that can be sent directly to the patient (e.g., to the patient's smartphone or computer) or to a clinician via the connectivity module 92.

As will be described in more detail below, one or more cloud-connected devices, such as one or more super-computers, may process patient-specific data, including data captured by the implantable medical device, and may perform ML and/or AI algorithms in analyzing the data. The data may include very large sets of data captured over long periods of time, such as data captured continuously by the implanted device over a long period of time. In some examples, one or more such devices may process data provided from the implantable medical device (or other data), whether received directly from the implantable medical device or alternatively from the external device 70, and may provide feedback to the external device 70 to optimize performance of local ML or AI processing at the external device 70.

The external device 70 includes various I/O devices that can be used to receive information from a user or provide information to a user. A microphone 93 may be used to receive voice commands, voice inputs, or sound inputs at the external device 70. In some examples, voice commands, sound environment recordings, breathing sounds, heart valve sounds, and other respiratory sounds such as cough, wheezing, labored breathing, pneumonia, and the like, may be recorded. In various implementations, voice commands can be processed and or stored by the implantable medical device or by the external device 70. In some examples, the implantable device can communicate a voice command to the external device (or vice versa) by long-distance telemetry (e.g., via MICS or BLE), and the external device 70 may process the voice commands, or in some cases communicate the voice commands to a cloud connected device (or other device) via the connectivity module 92.

An audio output module 94 can include one or more of a speaker or buzzer, for example, and can provide one or more of beeps or voice commands, according to various implementations. In some examples, the audio output module can provide features such as range finding when establishing a Galvanic communication link (e.g., by providing audible feedback so the user is aided in optimally positioning or aligning the external device to communicate with the implantable medical device.

A visual output module 95 can include one or more light-emitting diodes (“LED's”) that can provide status information. The one or more LED's can provide a status indication of, for example, external device battery life, implantable medical device battery life, galvanic communication link, faults, or status indicators, such as patient health status (e.g., presence of AF, arrhythmias, or no health concerns detected), to list just a few examples. The visual output module 95 may also include a graphical user interface (“GUI”) with an LCD screen (e.g., graphic text, black/white, or full color LCD), according to some implementations. The LCD screen may be a touch screen receptive to touch inputs in some implementations, or may provide soft keys for patient input or clinician input. The GUI can provide feedback and information to the patient on their health status, instructions from the physician, and cloud-based automated directions that may be prescribed by the clinician, according to various implementations. In some examples, patient input may be received via the GUI, including, for example input related to observed health conditions, such as a syncope event, dizziness, or not feeling well, or for entering information such as a daily activity log relating to activities such as eating, sleeping, exercising, or compliance with medication requirements.

The external device 70 can include one or more sensors 96, such as an accelerometer sensor or piezo element, temperature sensor, a weight measurement sensor, or a snoring sensor. The one or more sensors 96 may be used to detect and record the patient's body position, motion, or alignment of the external device 70 relative to the implanted device, for example, or to record the patient's temperature, weight, or indications of snoring.

The external device can optionally include a surface ECG measurement module 97, and auxiliary ECG electrodes 98 to record a surface ECG signal. FIG. 8B, described above, showed an example of an external device 82 that included auxiliary ECG measurement electrodes 84a, 84b. The auxiliary ECG measurement electrodes 84a, 84b of FIG. 8B were shown extending from the external device via wires, and in some examples electrodes 98 may similarly extend away from the external device 70, but in other implementations one or more of the electrodes 98 may be included on a bottom external surface of the external device 70.

In various implementations, the external device 70 may record, using the surface ECG measurement module 97 and auxiliary ECG electrodes 98 a patient's ECG signal and may compare it to the ECG signal recorded by the implantable medical device. In various implementations, the external device 70 may use such comparisons to detect issues with the implantable medical device, such as with the ECG measurement circuit of the implantable device. In some examples, the surface ECG measurements obtained by the external device 70 may have multiple vectors, which may provide a greater diagnostic ECG data. In some examples, more than two auxiliary ECG electrodes 98 can be used. For example, three electrodes 98 (or more) can be used in some implementations to provide two or more vectors. In some examples, a first vector may be orthogonal to a second vector. As shown with reference to FIG. 8B, auxiliary ECG wires can be used (e.g., by the patient) to record a much wider vector than would be possible for electrodes on the surface of the external device 70. A potential benefit of having multiple ECG surface electrode vectors and wider vectors may be that a larger volume of ECG information may be collected for the patient, which may enable deeper levels of analysis in some cases. This may helpful, for example, in cases where there is concern about the patient's condition, or where more diagnostic data is needed. In some examples, such additional ECG recording may be performed to maintain a patient trend. In some examples, such additional ECG recording may be performed to compare with results from the implantable ECG monitor.

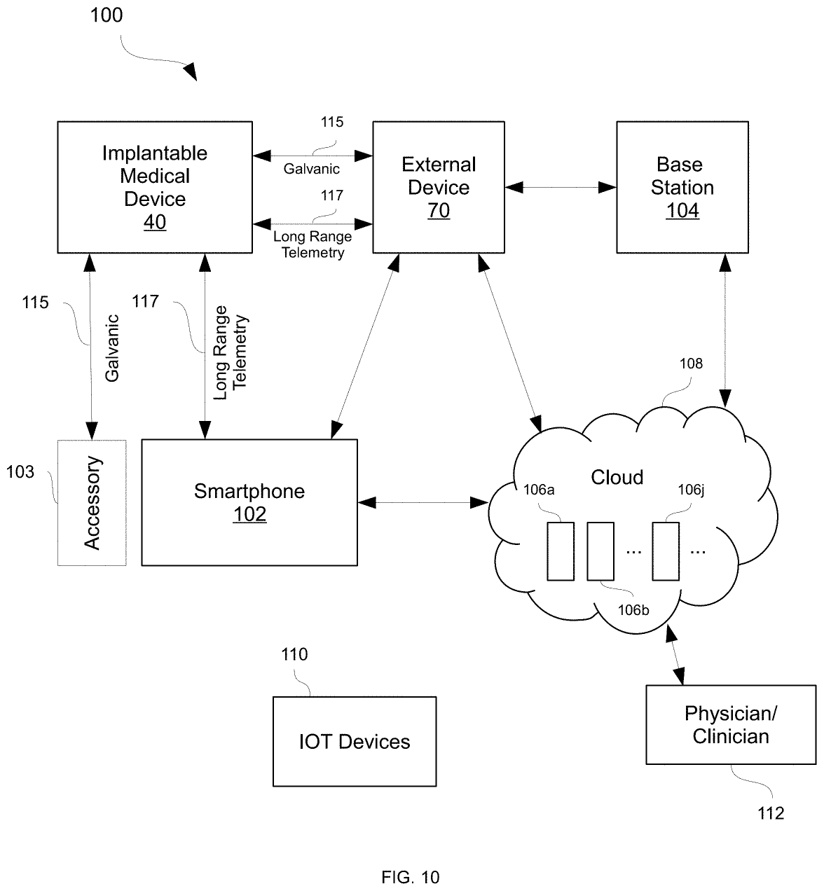

FIG. 10 is a system diagram of an environment 100 that includes an implantable medical device 40, an external device 70, a smartphone 102, a base station 104, cloud-connected computing devices 106, cloud storage 108, and various Internet of Things (IOT) devices 110, in accordance with one or more implementations. The implantable medical device 40 can communicate with the external device 70 by a Galvanic communication link 115. As described above herein, the Galvanic communication link 115 may be used for fast, efficient low-power transfer of large amounts of recorded physiological signal data from the implantable medical device 40 to the external device 70. For example, the implantable device 40 may continuously record physiological signal data (e.g., ECG, cardiac auditory signal, an indication of patient motion, an indication of patient respiration, an indication of patient temperature, an indication of patient pressure (e.g., blood pressure), cardiac acoustic or cardiac mechanical signals, an indication of body tissue impedance, or other physiological signals) and store it in memory of the implantable device, and then periodically (e.g., daily, weekly, monthly) upload the data over the Galvanic communication link 115 to the external device. Because the power transmission requirements for the implantable device with the Galvanic communications link 117 forming the long-range telemetry are very low compared to RF telemetry power requirements, for example, the implantable device 40 may be able to capture, store and upload much larger collections of physiological signal data than was previously possible using RF telemetry systems to upload data from an implantable device. Also, because transmission speeds with the Galvanic communications link 117 forming the long-range telemetry may be extremely fast, the time needed to upload large amounts of data may be relatively brief.

In some implementations where the implantable device 40 includes long-range telemetry functionality (e.g., such as module 55 of FIG. 5), the implantable medical device 40 can communicate with the external device 70 or the smartphone 102 by a long-range telemetry protocol 117, such as BLE or MICS, for example. Long-Range telemetry may be used for alerts or situations where large amounts of data may not need to be sent. Alerts or warnings may be sent via long-range telemetry, in some implementations. The smartphone 102 (which may correspond to the patient's smartphone) can provide a user interface to display messages, instructions, trends, diagnostic information, status information, alert information, and can be used to receive user input. The external device 70 and the smartphone 102 may communicate using BLE, for example, or over another network. While a smartphone 102 is depicted in FIG. 10, in some implementations the smartphone 102 may be replaced by a smart-watch or other wearable device, by a tablet computing device, by a laptop computing device, by an internet appliance, or by another appropriate device, for example.

In some examples, an accessory 103 to the smartphone 102, such as a smartphone case or a plug-in adapter configured with two or more surface communication electrodes and a Galvanic communication module (e.g., similar to the module 53 of FIG. 5 or the module 88 of FIG. 9), may be used to permit the implantable medical device 40 to communicate with the smartphone 102 by a Galvanic communication link 115, for example if the patient holds the accessory 103 over the implantable medical device 40 against the skin of the patient in a manner similar to that shown in FIG. 8A with the external device 80. The accessory 103 may communicate with the implantable device 40 via a Galvanic communication link 115, for example, and may communicate with the smartphone 102 via BLE, for example, effectively permitting the implantable device 40 to communicate with the smartphone 102 without requiring a long-range telemetry module at the implantable device 40, according to some implementations.

The external device 70 may communicate data to (or receive data from) the cloud 108 or to cloud-connected devices 106 (e.g., one or more super-computers 106a, research stations 106b, desktop computer 106j, and the like) via one or more networks, such as a one or more wired or wireless networks (e.g. WiFi, wired internet, one or more local area networks (LANs) or wide area networks (WANs), or other communications or network techniques. In some implementations, super-computer 106a may receive the data uploaded from the external device 70, after having received it from the implanted device 40, and the super-computer 106a may process the data using ML algorithms and/or AI algorithms. The super-computer 106a may then transmit results of the analysis to the external device 70 or to the smartphone 102, for example. A physician or clinician 112 may similarly receive the results of the analysis, and may prescribe changes to the patient's treatment plan, according to some implementations, which may be communicated to the external device 70 in some implementations.

Internet of Things devices 110, such as a weight scale, a blood pressure cuff, a smart medication usage detection device, a motion detector, an appliance, or the like, may be used in the environment 100 with connectivity to the rest of the system (not shown, but generally using any appropriate communications technology), allowing data from one or more of these devices 110 to be integrated into the data set and used for patient health trending, diagnosis, and machine learning.

The base station 104 may provide connection to the internet or cloud and may also be a place to store and charge the external device 70. In some implementations, the base station 104 may include a user interface to provide information to the patient, and for the patient to enter data such as symptoms, medication compliance, and the like.

Patients who have a prior history of AF may use an external mobile app (e.g., an app associated with the smartphone 102, the external device 70, or the base station 104) to store and manage AF-related medication information. As described above herein, patient's with the implantable medical devices discussed herein may benefit from continuous recording of the patient's ECG signal by the implantable device, where the continuously recorded ECG signal is stored in memory of the implantable device, and later uploaded to a device external of the body of the patient, for example to one or more of the external device 70, the smartphone 102, the base station 104, or one or more other computing devices, such as a cloud-connected super-computer. Further analysis of the continuously recorded ECG data may be performed, in various implementations, by any of the aforementioned devices. In some examples one or more ML algorithms or AI algorithms may process the continuously recorded ECG data of the patient, in some cases in conjunction with population data, and may use the results of the analysis to optimize future AF management (e.g., via medication adjustments, or via adjustments to one or more parameters of the implantable medical device) for the patient. In some examples, the algorithm may compare a data set to a template derived from population data, and may make a prediction or a detection based at least in part on the comparison. In some examples, such analysis can occur at a cloud-connected super-computer or other computing device, in some examples such analysis can occur at the external device 70, and in some examples a portion of the analysis may be performed at the external device 70 and a portion of the analysis may be performed at a cloud-connected super-computer or other computing device, where the results from the cloud-connected super-computer or other computing device analysis may be used as well by the external device 70 in its analysis.

In some implementations, the implantable medical device 40 can include an AF detection algorithm, and can analyze the continuously recorded ECG signal, at the implantable medical device 40, and determine an AF burden (e.g., a total duration of time that the patient stays in AF per day). If the AF burden exceeds a predetermined threshold, for example, the implantable medical device 40 can provide or transmit an alert to the patient (e.g., via a buzzer or via a message to the patient's smartphone 102), to a physician or clinician, or to the patient and the physician or clinician.

Initially at time of implant, the physician may prescribe a frequency of data upload from the implantable medical device 40 to the external device 70 based on the patient's history or on a recent or current measurement of the patient's AF burden, for example. As an example, if the patient is in persistent AF, or is in AF for about five hours or more per day, the physician may request that the patient upload the continuously recorded ECG data from the implantable medical device 40 to the external device 70 daily. Conversely, if the patient is in paroxysmal AF, the physician may request that the patient upload the continuously recorded ECG data from the implantable medical device 40 to the external device 70 only monthly. In some examples, the patient's smartphone 102 or the external device 70 may receive an alert indicating that it is time to upload data from the implantable medical device 40 to the external device. The patient may position the external device 70 over the implanted medical device 40, and the data may be quickly uploaded from the implantable medical device 40 to the external device 70.

As mentioned previously, additional analysis of the continuously recorded ECG signal information can be performed outside of the implantable medical device 40. The discussions that follow will assume that such analysis occurs at a remote computing device with access to the cloud, such as a super-computer with access to the cloud, but in other implementations the analysis could alternatively be performed at the external device 70. The external device 70, having received the uploaded data from the implantable medical device 40, may transmit the data to a cloud-based storage location, according to various implementations. A computing device or analysis platform may analyze the continuously recorded ECG signal information to perform an analysis to determine an AF burden (performed externally of the implantable device, with the benefit of additional computing power and additional data).

The results or information from the external algorithm can be used to optimize one or more settings of the internal AF detection algorithm within the implantable device 40 to improve accuracy, and such results may be communicated to the external device 70 which may pass along the information to the implantable device 40, for example.

In various implementations, machine-learning algorithms (or AI algorithms) may be performed locally or remotely. Local machine learning algorithms can be performed by the implantable medical device 40 or by the external device 70 (which may both be referred to as local processing, for purposes of this discussion) to implement machine learning relating to the specific patient, where the ML algorithms may learn from any of the sensors or devices discussed above, and from patient provided input of symptoms and daily activities, according to some implementations. Remote machine learning algorithms, which may be cloud-based, may provide processing analysis that goes deeper than the local analysis (e.g., may analyze AF burden at a deeper level, or may additionally analyze for other arrhythmias), and may use data from a broader patient population, according to some implementations. In some examples, the algorithm may compare a data set to a template derived from population data, and may make a prediction or a detection based at least in part on the comparison.

In some examples, the cloud-based based machine learning may be a standalone analysis, and in some implementations the cloud-based based machine learning can be coupled with the local machine learning to augment or refine local algorithms, for example. In some examples, local machine learning algorithms may execute periodically or continuously in real-time, while the cloud-based machine learning remote algorithm processing may provide deeper analysis on an occasional or less frequent basis, for example. In various implementations, one or more of the analysis platforms (local, remote, or both) can send alerts to the patient or to the physician or clinician, such as alerts based on AF burden thresholds and instances or periods where the threshold is exceeded. Alerts to the physician could be via a physician web portal, emails, texts or automated phone calls, while alerts to the patient could be via a buzzer or speaker message from the implantable device, or by email, text or automated phone call to the patient's smartphone 102, or by an indication from the external device 70.

In some examples, the physician can make an adjustment of medication based on the AF burden, for example, such as reducing an antiarrhythmic and/or anticoagulation medication when the AF burden is low, or increasing such medication when the AF burden is high.

Some machine learning based AF management algorithms may take as inputs to the algorithm, for example, a change in the patient's medication, a change in patient AF burden, or other clinical outcome (e.g., stroke, bleeding occurrence). In some examples, the machine-learning algorithm can use both patient-specific information and population-based data to predict upcoming onsets or periods where the patient may be in AF and deliver an alert to warn the patient of a predicted upcoming AF onset or period. In some examples, the algorithm may compare a data set to a template derived from population data, and may make a prediction or a detection based at least in part on the comparison. In some examples, the algorithm can generate recommendations for the physician or clinician regarding a recommended frequency of data upload, or a recommended change to an existing frequency. In some examples, the algorithm can generate recommendations for the physician or clinician regarding a recommended medication change or dosage change for the patient. In some examples, machine learning can provide predictive information on AF management, including suggesting earlier changes to medication doses or decision-making criteria for interventional procedures.

The machine learning algorithms may, in some implementations, use both patient-specific information and population-based data to generate recommendations of medication changes for the physician to consider and prescribe. Some of the discussions above were focused on ML algorithms, but in general the discussions may also apply to AI algorithms, for example.

Beyond, or in additional to, AF, patients may use the devices, systems, and techniques discussed herein to detect, treat and manage other conditions or events, such as syncope, stroke, or myocardial infarction. In some examples, the implantable medical device 40 may include a detection algorithm or predictive algorithm or analysis algorithm regarding one or more of these conditions or events. In some examples, the external device 70 may include a detection algorithm or predictive algorithm or analysis algorithm regarding one or more of these conditions or events, and in some cases one or more of the algorithms may be a ML algorithm or an AI algorithm. In some examples, a remote computing device, such as a super-computer with access to the cloud may include a detection algorithm or predictive algorithm or analysis algorithm regarding one or more of these conditions or events, and in some cases one or more of the algorithms may be a ML algorithm or an AI algorithm.

A machine learning based syncope management algorithm can take as inputs, for example, the continuously recorded ECG and/or EEG signal information, recorded motion information (e.g., continuously recorded or periodically recorded), recorded temperature information (e.g., continuously recorded or periodically recorded), time information (e.g., times or timing information associated with any of the other inputs), and patient-reported occurrences of pre-syncopal events and syncopal events, according to some implementations. In some examples, the machine-learning algorithm can use both patient-specific information and population-based data to predict upcoming syncope events and deliver an alert to warn the patient of a predicted upcoming syncopal event. In some examples, the algorithm may compare a data set to a template derived from population data, and may make a prediction or a detection based at least in part on the comparison. In some examples, the algorithm can generate recommendations for the physician or clinician regarding a recommended frequency of data upload, or a recommended change to an existing frequency. In some examples, the algorithm can generate recommendations for the physician or clinician regarding a recommended medication change or dosage change for the patient. In some examples, machine learning can provide predictive information on syncope management, including suggesting earlier changes to medication doses or decision-making criteria for interventional procedures.

In general, machine-learning algorithms executing in the cloud or executing on machines with access to data stored in the cloud may continuously learn and improve prediction of upcoming events, detection of past events, which may result in generally more positive outcomes. Such learning can be used to modify real-time detection at the implantable medical device 40 or processing at the implantable medical device 40 or at the external device 70. In some examples, the machine learning algorithms may provide patient-specific improvements in detection of syncope or other events, for example.

A machine learning based stroke management algorithm can take as inputs, for example, AF burden over a predetermined past duration of time, medication information (e.g., patient's prescribed dosage of anticoagulation medication), patient reported clinical events or outcomes (e.g., suspected stroke occurrence), physician input of clinical events or outcomes (e.g., stroke occurrence), and medication adherence information, according to some implementations. The machine-learning algorithm can use both patient-specific information and population-based data to predict upcoming stroke events and deliver an alert to warn the patient of a predicted upcoming stroke event. In some examples, the algorithm may compare a data set to a template derived from population data, and may make a prediction or a detection based at least in part on the comparison. In some examples, the algorithm can generate recommendations for the physician or clinician regarding a recommended frequency of data upload, or a recommended change to an existing frequency. In some examples, the algorithm can generate recommendations for the physician or clinician regarding a recommended medication change or dosage change for the patient. In some examples, machine learning can provide predictive information on stroke management, including suggesting earlier changes to medication doses or decision-making criteria for interventional procedures.

A machine learning based ischemia or myocardial infarction (“MI”) management algorithm can take as inputs, for example, the continuously recorded ECG signal information, recorded motion information (e.g., periodically or continuously recorded), recorded temperature information (e.g., periodically or continuously recorded), time information (e.g., times or timing information associated with any of the other inputs), patient-reported MI related symptoms (e.g., chest pain, angina), and physician-determined diagnoses of clinical of MI, ischemia, or both MI and ischemia. In some examples, the machine-learning algorithm can use both patient-specific information and population-based data to predict upcoming ischemic or MI events and deliver an alert to warn the patient of a predicted upcoming ischemic or MI event. In some examples, the algorithm may compare a data set to a template derived from population data, and may make a prediction or a detection based at least in part on the comparison. In some examples, the algorithm can generate recommendations for the physician or clinician regarding a recommended frequency of data upload, or a recommended change to an existing frequency. In some examples, the algorithm can generate recommendations for the physician or clinician regarding a recommended medication change or dosage change for the patient. In some examples, machine learning can provide predictive information on MI management, including suggesting earlier changes to medication doses or decision-making criteria for interventional procedures (e.g., stenting or coronary artery bypass graft).

In general, machine-learning algorithms discussed herein can be heuristic or statistical, according to some implementations. Heuristic algorithms may include artificial neural networks, and may include fuzzy logic approaches. Statistical algorithms may include Kalman filters, predictive filters, or both Kalman filters and predictive filters, according to some implementations.

In some examples, the implantable medical device may detect respiration or respiratory rate by measuring tissue impedance, and in some cases can use readings from the accelerometer in conjunction with the respiration to make predictions or detections. For example, a change in respiratory rate without an accompanying change in whole-body acceleration may be indicative of a change in the pulmonary condition of the patient, such as an onset or an increase in pulmonary edema, which could require medical attention.

Changes in the whole body motion of the patient may indicate exercise or exertion, and can be monitored by the implantable medical device to determine compliance with a prescribed exercise regimen. Simultaneous recordings of ECG and whole body motion can be used to determine correlations between the metabolic demand and the response of the cardiovascular system, according to some implementations. Deviations from predetermined norms can be used to detect abnormalities such as sick sinus node syndrome, exercise induced tachycardia, or paroxysmal atrial fibrillation, according to some implementations.

In some examples, changes in the whole body motion of the patient may indicate periods of rest. This can be useful in assessing sleep, for example, such as if an expected change the heart rate and heart rate variability is expected but not observed during sleep. In such cases, an adjustment to a medication may be recommended. In some examples, changes in the whole body motion of the patient may indicate falls, which can be a concern for the elderly population. If it is determined that a fall has occurred, an alert may be provided to the patient with a required response to determine if the patient is conscious, and if the patient needs help. If the patient needs help or if no response is received, an alert may be sent to one or more family members, caregivers, and medical professionals.

While the communications signals described above with reference to communications between the implantable medical device 40 and the external device 70 were described as being transmitted without modulation onto a carrier, on some alternative examples, the communications signals may be modulated onto a carrier waveform for transmission, and demodulated from the carrier waveform after reception. In some examples, the carrier waveform may be used as a narrow bandpass filter to detect the carrier signal in the external device electronics. Options for modulation can include amplitude modulation, pulse-position modulation, pulse amplitude modulation, and pulse-width modulation.

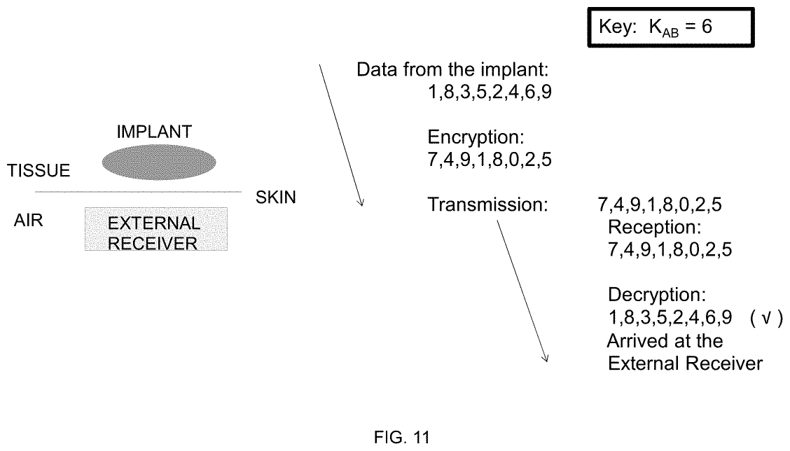

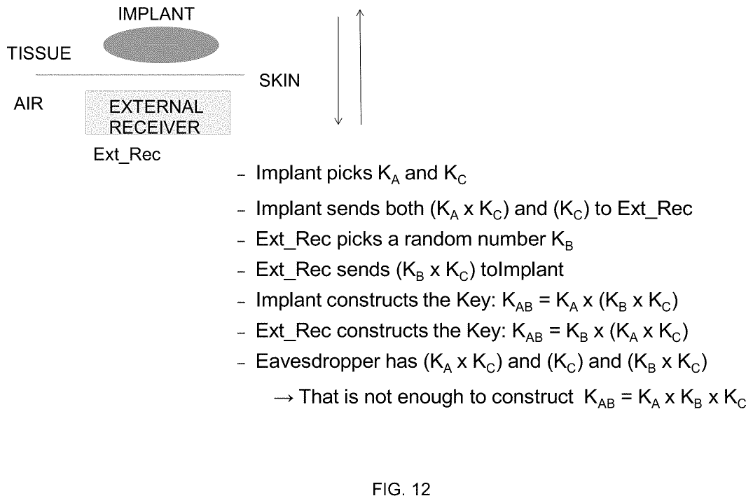

Referring again to the encoder 11 of FIGS. 2-3, the encoder 11 may optionally encrypt data to be transmitted (e.g., to the external device 70), so that if the data is intercepted that it may not be interpreted, according to some implementations. Accordingly, the data can be decrypted when it is received at a receiver (e.g., the external device 70). Possible encryption techniques can include a simple key encryption (see FIG. 11 for an example of a simple key encryption technique), a public key encryption (see FIG. 12 for an example of a public key encryption technique), and Secure Socket Layer (“SSL”) digital certificate. The descriptions that follow will assume transmission of data from the implantable medical device 40 to the external device 70, but other examples can include the implantable device 40 transmitting data to another device, or the external device 70 transmitting data to the implantable device 40 or to another device.

FIG. 11 shows an example operation of a simple key encryption system, in accordance with one or more implementations. A secret key, K_{AB}, is shared between the implantable medical device 40 and the external device 70. Each data byte that is sent is increased by the amount of K_{AB}, which has the value 6 in this example. Upon receipt of the transmitted data, the received data value is reduced by the same amount to recover the original data. This method remains viable as long as the key remains secret.

An alternative implementation is illustrated in FIG. 12, which is known as public key encryption. FIG. 12 shows an example operation of a public key encryption system, in accordance with one or more implementations. In the example of FIG. 12, the implantable device 40 picks two random numbers, K_{A} and K_{C}. Next, the implantable device 40 sends both (K_{A}×K_{C}) and (K_{C}) to external device 70. The external device 70 picks a random number, K_{B}, and then the external device 70 sends (K_{B}×K_{C}) to the implantable device 40. The implantable device 40 constructs the key K_{AB}=K_{A}×(K_{B}×K_{C}), and the external device 70 constructs the key K_{AB}=K_{B}×(K_{A}×K_{C}). Any eavesdropper may have the products (K_{A}×K_{C}) and (K_{B}×K_{C}), as well as the public key (K_{C}), for example, but may still lack necessary information to construct the private key K_{AB}=K_{A}×(K_{B}×K_{C}). To prevent the reverse calculation using division, modulo arithmetic can be used for the calculation, as division is not a unique function in modulo arithmetic.

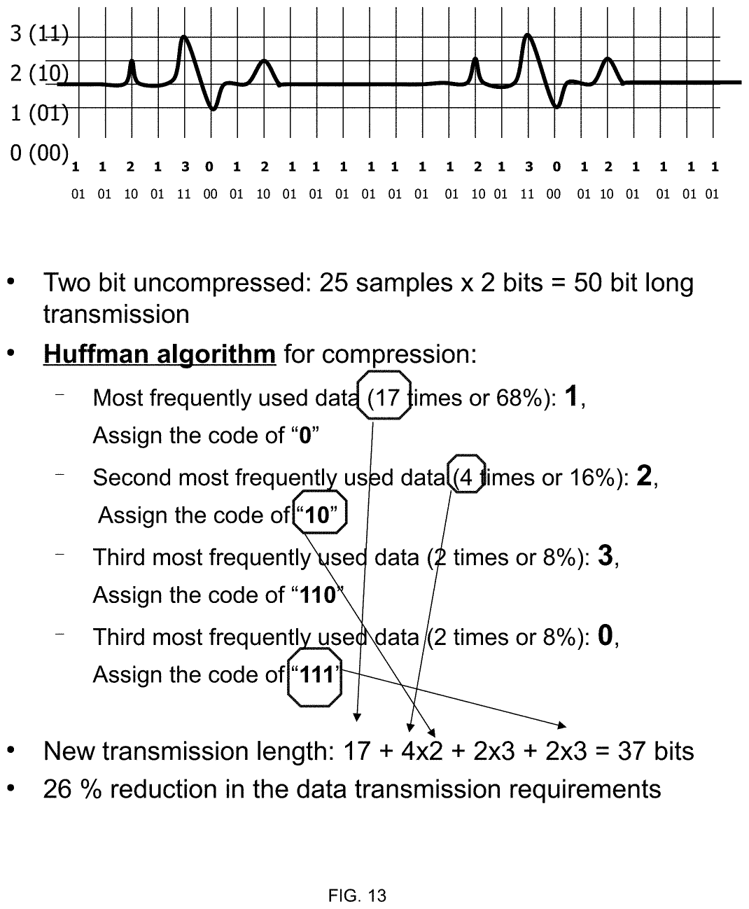

Regarding data compression techniques that can be used to compress data (e.g., captured physiological signal data) such that additional data may be stored in a given amount of memory, or to reduce an amount of data to be transmitted, several techniques can be used. For example, data compression techniques can include the Huffman algorithm, which is a lossless data compression technique. FIG. 13 shows an example data compression operation using a Huffman algorithm, in accordance with one or more implementations. The example of FIG. 13 uses a data set with 2-bit resolution. In some implementations, the data can initially be stored without compression and analyzed periodically. The most frequently occurring measurement, “01” in this example, can be assigned the shortest transmission frame, “0” in the example. The second-most frequently occurring measurement can be assigned a next shortest transmission frame, “10” in this example. In this manner, the overall transmission length can be reduced significantly, according to some implementations. ECG data and respiration data may be particularly suitable for this type of data compression, as both may contain long intervals where the signal typically remains steady.

In various implementations, this type of data compression can be done at different stages of operation of the device. In some implementations, the implantable medical device 40 can store the raw data for a period of time, such as one minute, and can then compress it to reduce the amount of memory needed for storage of the data. Alternatively, the device 40 can store all the data in its raw format, and wait to do the compression immediately before transmitting the data from the device.

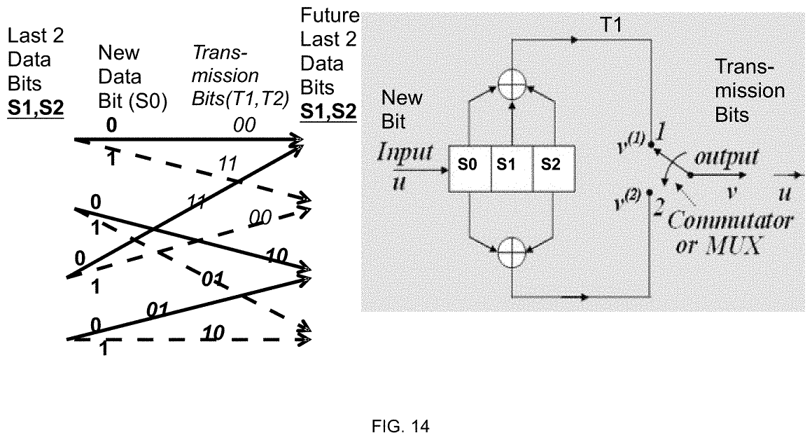

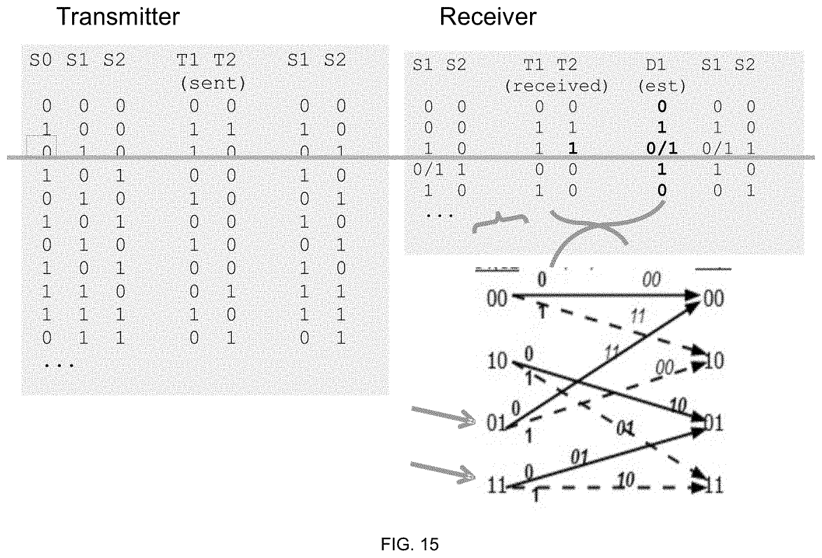

Any transmission can be prone to error, and in some cases detection of errors may require a re-transmission of the data, which in turn can prolong the data transmission session and cause more battery power to be used. One technique that can be used to mitigate this issue is to use forward error correction schemes. FIGS. 14 and 15 provide examples, and will be discussed below.

FIGS. 14 and 15 show example operations of an encoder using a forward error correction code, in accordance with one or more implementations. FIG. 14 shows an encoding system where the data that is being transmitted is a function of the bit that is being sent, as well as the two bits that were previously sent. Because the possible transmission patterns are limited, errors in detection can be noticed and can be corrected by referring to the bits that were sent before and after the bit with reception error. This is further illustrated in FIG. 15, where an error that occurred during the transmission of the third line was detected and corrected at the end of the fourth line.

Continuous sampling and recording of one or more physiological signals, over extended periods of time, can be memory intensive at the implantable medical device 40. For example, at a 125 Hz data sample rate and storing 1 byte of data for each sample, 10.8 megabytes may be required to store one day's worth of continuously recorded data, according to some implementations. At a 125 Hz data sample rate and storing 1 byte of data for each sample, 75.6 megabytes may be required to store one week's worth of continuously recorded data, according to some implementations. At a 125 Hz data sample rate and storing 1 byte of data for each sample, 324 megabytes may be required to store one month's worth of continuously recorded data, according to some implementations.

Similarly, at a 125 Hz data sample rate and storing 2 bytes of data for each sample, 21.6 megabytes may be required to store one day's worth of continuously recorded data, 151.2 megabytes may be required to store one week's worth of continuously recorded data, and 648 megabytes may be required to store one month's worth of continuously recorded data, according to some implementations. If it is desired to store 4 bytes of data for each sample at 125 Hz data sample rates, the numbers above in this paragraph may be doubled, for example.

In a first general aspect, a method monitoring a physiological signal of a patient, includes sensing, from a subcutaneous implant location with an implantable medical device, a physiological signal of the patient. The method also includes capturing, by the implantable medical device, the physiological signal and storing data representing the captured physiological signal to memory of the implantable medical device. The method further includes transmitting, by the implantable medical device for receipt by a device external of the body of the patient, the data representing the captured physiological signal via a Galvanic communications link between the implantable medical device and the device external of the body of the patient. Body tissue of the patient serves as a conductive medium for the transmission of the data representing the captured physiological signal.

Various implementations can include one or more of the following. The data representing the captured physiological signal may be transmitted without using radio frequency telemetry, and without using a radio frequency antenna. The data representing the captured physiological signal may be transmitted without modulating the data representing the captured physiological signal onto a carrier wave. A first electrode and a second electrode may be used to capture the physiological signal, and the first electrode and the second electrode may further be used to transmit the data representing the captured physiological signal via the Galvanic communications link. A first electrode and a second electrode may be used to capture the physiological signal, and at least one of the first electrode and the second electrode may further be used to transmit the data representing the captured physiological signal via the Galvanic communications link. The memory of the implantable medical device may include a memory device with at least five megabytes of storage capacity. A first electrode and a second electrode may be used to capture the physiological signal, at least one of the first electrode and the second electrode may further be used to transmit the data representing the captured physiological signal via the Galvanic communications link, and the data representing the captured physiological signal and transmitted via the Galvanic communications link may include at least five megabytes of data. The data representing the captured physiological signal and transmitted via the Galvanic communications link and including at least five megabytes of data may be transmitted via the Galvanic communications link over a single communications session. The implantable medical device may continuously sense and capture the physiological signal over a period of at least four hours, and data stored to the memory of the implantable medical device may represent the continuously captured physiological signal over the period of at least four hours, and the data transmitted via the Galvanic communications link may include the data that represents the continuously captured physiological signal over the period of at least four hours. The method may further include detecting, by the implantable medical device, an abnormality in the captured physiological signal, and transmitting, via radio frequency telemetry for receipt by a second device external of the body of the patient, data representing the captured physiological signal and the abnormality. The device external of the body of the patient and the second device external of the body of the patient may be the same device.

In a second general aspect, a system for monitoring a physiological signal of a patient includes an implantable medical device and a device external of the body of the patient. The implantable medical device is configured to sense, from a subcutaneous implant location, a physiological signal of the patient, and capture the physiological signal and store data representing the captured physiological signal to memory of the implantable medical device. The implantable medical device is also configured to transmit, for receipt by the device external of the body of the patient, the data representing the captured physiological signal via a Galvanic communications link between the implantable medical device and the device external of the body of the patient, where body tissue of the patient serves as a conductive medium for the transmission of the data representing the captured physiological signal. The device external of the body of the patient is configured to receive, from the implantable medical device, the data representing the captured physiological signal via the Galvanic communications link between the implantable medical device and the device external of the body of the patient. The device external of the body of the patient is also configured to analyze the data representing the captured physiological signal using an algorithm selected from the group consisting of a machine learning algorithm and an artificial intelligence algorithm. The device external of the body of the patient is further configured to provide, to the implantable medical device based on a result from the analysis of the data representing the captured physiological signal, a parameter that the implantable medical device can use to adjust an operation on the implantable medical device.

Various implementations can include one or more of the following. The data representing the captured physiological signal may be transmitted without using radio frequency telemetry, and without using a radio frequency antenna. The data representing the captured physiological signal may be transmitted without modulating the data representing the captured physiological signal onto a carrier wave. The implantable medical device may use a first electrode and a second electrode to capture the physiological signal, and may use the first electrode and the second electrode to transmit the data representing the captured physiological signal via the Galvanic communications link. The implantable medical device may include a first electrode and a second electrode that are used to capture the physiological signal, and the implantable medical device may use at least one of the first electrode and the second electrode to transmit the data representing the captured physiological signal via the Galvanic communications link. The memory of the implantable medical device may include a memory device with at least five megabytes of storage capacity. The implantable medical device may include a first electrode and a second electrode that are used to capture the physiological signal, and the implantable medical device may use at least one of the first electrode and the second electrode to transmit the data representing the captured physiological signal via the Galvanic communications link, and the data representing the captured physiological signal and transmitted via the Galvanic communications link may include at least five megabytes of data. The data representing the captured physiological signal and transmitted via the Galvanic communications link and including at least five megabytes of data may be transmitted via the Galvanic communications link over a single communications session. The implantable medical device may continuously sense and capture the physiological signal over a period of at least four hours, and data stored to the memory of the implantable medical device may represent the continuously captured physiological signal over the period of at least four hours, and the data transmitted via the Galvanic communications link may include the data that represents the continuously captured physiological signal over the period of at least four hours. The implantable medical device may be further configured to detect an abnormality in the captured physiological signal, and transmit, via radio frequency telemetry for receipt by a second device external of the body of the patient, data representing the captured physiological signal and the abnormality. The device external of the body of the patient and the second device external of the body of the patient may be the same device. The device external of the body of the patient may be further configured to receive an input that specifies a clinical event, and the analyzing the data representing the captured physiological signal may include consideration of the received input that specifies a clinical event. The input that specifies a clinical event may be received from the patient, or from a physician.

In a third general aspect, a system for monitoring a physiological signal of a patient includes an implantable medical device, a device external of the body of the patient, and a remote computing device. The implantable medical device is configured to sense, from a subcutaneous implant location, a physiological signal of the patient, and capture the physiological signal and store data representing the captured physiological signal to memory of the implantable medical device. The implantable medical device is also configured to transmit, for receipt by the device external of the body of the patient, the data representing the captured physiological signal via a Galvanic communications link between the implantable medical device and the device external of the body of the patient. Body tissue of the patient serves as a conductive medium for the transmission of the data representing the captured physiological signal. The device external of the body of the patient is configured to receive, from the implantable medical device, the data representing the captured physiological signal via the Galvanic communications link between the implantable medical device and the device external of the body of the patient. The device external of the body of the patient is also configured to transmit, for receipt by the remote computing device, the data representing the captured physiological signal. The remote computing device is configured to receive, from the device external of the body of the patient, the data representing the captured physiological signal, and analyze the data representing the captured physiological signal using an algorithm selected from the group consisting of a machine learning algorithm and an artificial intelligence algorithm. The remote computing device is also configured to provide, to the implantable medical device based on a result from the analysis of the data representing the captured physiological signal, a parameter that the implantable medical device can use to adjust an operation on the implantable medical device.

Various implementations can include one or more of the following. The data representing the captured physiological signal may be transmitted without using radio frequency telemetry, and without using a radio frequency antenna. The data representing the captured physiological signal may be transmitted without modulating the data representing the captured physiological signal onto a carrier wave. The implantable medical device may use a first electrode and a second electrode to capture the physiological signal, and may use the first electrode and the second electrode to transmit the data representing the captured physiological signal via the Galvanic communications link. The remote computing device may be configured to, using the algorithm selected from the group consisting of a machine learning algorithm and an artificial intelligence algorithm, use data associated with a population of patients, and the remote computing device may be further configured to provide, to the implantable medical device based on a result from the analysis of the data representing the captured physiological signal and the data associated with the population of patients, a parameter that the implantable medical device can use to adjust an operation on the implantable medical device. The implantable medical device may include a first electrode and a second electrode that may be used to capture the physiological signal, and the implantable medical device may use at least one of the first electrode and the second electrode to transmit the data representing the captured physiological signal via the Galvanic communications link. The memory of the implantable medical device may include a memory device with at least five megabytes of storage capacity. The implantable medical device can include a first electrode and a second electrode that can be used to capture the physiological signal, and the implantable medical device can use at least one of the first electrode and the second electrode to transmit the data representing the captured physiological signal via the Galvanic communications link, and the data representing the captured physiological signal and transmitted via the Galvanic communications link can include at least five megabytes of data. The data representing the captured physiological signal and transmitted via the Galvanic communications link and including at least five megabytes of data can be transmitted via the Galvanic communications link over a single communications session. The implantable medical device can continuously sense and capture the physiological signal over a period of at least four hours, and data stored to the memory of the implantable medical device can represent the continuously captured physiological signal over the period of at least four hours, and the data transmitted via the Galvanic communications link can include the data that represents the continuously captured physiological signal over the period of at least four hours. The implantable medical device can be further configured to detect an abnormality in the captured physiological signal, and transmit, via radio frequency telemetry for receipt by a second device external of the body of the patient, data representing the captured physiological signal and the abnormality. The device external of the body of the patient and the second device external of the body of the patient may be the same device. The remote computing device may be further configured to receive an input that specifies a clinical event, and the analyzing the data representing the captured physiological signal can include consideration of the received input that specifies a clinical event. The input that specifies a clinical event can be received from the patient, or from a physician.

Invention can be used for the monitoring and treatment of many disease states, included by not limited to:

Cardiovascular diseases, such as arrhythmias, chronic heart disease and valvular disorders: For the monitoring of the cardiac arrhythmias, the ECG signals are monitored, recorded and analyzed by the implant, the external units and the cloud based processors. For the patients suffering from the chronic heart disease (CHS) or valvular disorders, monitoring of their cardiac output, heart rate and the status of the pulmonary edema may be needed, most of which can be accomplished using impedance plethysmography. Based on the location of the implant, and if necessary, using an optional pigtail electrode, one can monitor the tissue impedance and interpret the changes in the impedance as a change in the fluid content of the tissues where the electrical impedance is measured from. For example, transient changes in the tissue impedance would be interpreted to be due to heart beat and respiration. A low rate signal in the range of 5 to 15 beats per minute (bpm) would be due to respiration where the signals having a frequency of 25 bpm and higher would be due to heart beat. Signals that are changing much more slowly, such as in hours, would be due to the changes in the pulmonary edema while the fluid levels in the lungs change. An analog, or preferably a digital filter that is implemented in the implant, external device, such as a mobile phone, or in the cloud processor can extract the information described in this paragraph.

Kidney diseases, such as chronic kidney disease and volume overload: As the kidneys of the patients fail, the fluids and other toxins, such as electrolytes and urea begin to accumulate in the tissues of the patients. Although these patients usually receive dialysis treatment, the fluid and toxin levels do still accumulate in the body, especially between the hemodialysis sessions. As a matter of fact, many cardiac arrhythmias in this group of patients take place at the end of the weekend which is when the patients had not been dialyzed for almost 72 hours. Monitoring of the volume overload in these patients using the impedance monitoring scheme as described in the previous paragraph can be accomplished by the invention as described.

Neurological disorders: Patients with neurological disorders, such as stroke and Parkinsons, suffer from movement disorders, such as ataxia, tremors and bradykinesia. Using its internal sensor, such as the accelerometer, implant monitors the posture and the motion of the subject, and allows the correlation of the drug treatments, such as those using L-dopa, to the individual patient outcome. For example, the overuse of L-dopa should be avoided to minimize the adaptation of the tissue and to minimize the mood swings that the drug causes. By monitoring the patient outcome, dynamic adjustments to the drug dosage and the time of drug administration can be made. Other uses of the invention in patients with neurological disorders include the monitoring of the electroencephalogram (EEG) of the patients with epilepsy or those working on recovery from a brain injury.

Sleep disorders: Patients with obstructive sleep apnea suffer from reduced oxygenation of their peripheral tissues during apneic episodes. Using a reflective oxygenation sensor, the implant can monitor the perfusion levels in peripheral tissues and provide the feedback that is necessary for the adjustment of the therapy, such as a recommendation to increase pressure on the continuous positive airway pressure (CPAP) device.

Invention uses the machine learning algorithms for multiple purposes, included by not limited to:

Algorithm for the determination of the values of trigger thresholds based on historical population data: Immediately after the implantation of the device, there is no patient specific information that can be used for the triggering of the alarms based on the physiological information that is obtained from the patient. For example, it is now clear how much should the chest impedance be allowed to reduce for a given subject before the pulmonary edema warning should be issued. In that case, algorithm that is running on the implant, the external unit or the cloud would interpret the patient's data by using thresholds that are chosen based on the population based values, that are predetermined by the machine-learning algorithm, by selecting values that are common to individuals with similar medical characteristics, such age, body weight, body mass index, sex, disease state, co-morbidities and so on. This would allow the implant to be able to generate actionable information soon after the patient receiving the device. The machine learning algorithms that can be used for this purpose include, but not limited to, clustering, back propagation (aka Artificial Neural Networks), fuzzy networks, reinforcement learning and genetic algorithm.

Algorithm for the determination of the values of trigger thresholds based on patient's own data: Once a period of time has elapsed following the implantation of the device, the implant, the external unit and the cloud storage is populated patient specific information that can be used for the determination of the thresholds for triggering of the alarms. For example, when the tissue impedance that is below a given value, which requires a hospitalization of the subject due to pulmonary edema, or an increase in atrial fibrillation burden that causes the mobility of the subject to be limited would constitute patient specific triggers. The machine learning algorithms that can be used for this purpose include, but not limited to, predictive filters, unsupervised learning, Kalman filters, least squares, and error-driven algorithm, or a combination of these algorithms. Regardless of the method that was used for the selection of these trigger values, they can be determined by the algorithms that are running on the implant, the external unit or the cloud, and used for the continuous monitoring of the patients.

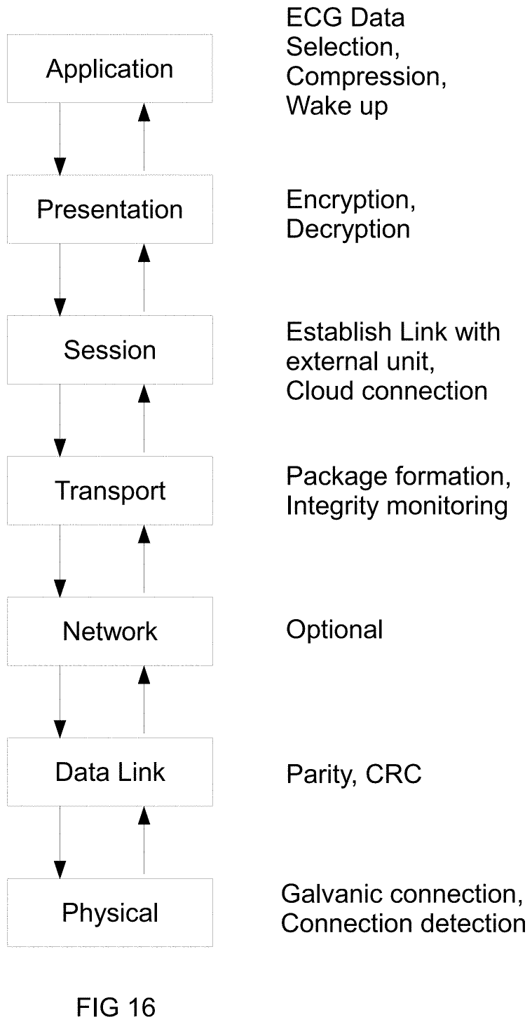

Data communications with the implant is done using a layered structure as in FIG. 16. The application layer provides the interface to the main algorithm that is running on the processor, and allows the selection of the dataset for compression. It may also allow the compression of the data as demonstrated in FIG. 13. The presentation layer provides the data encryption/decryption activity, as illustrated in FIGS. 11 and 12. Session layer would establish and maintain the link with the external units or the cloud based servers, as shown in FIG. 10. Transport layer allows the formation of individual packages for the transmission of large data sets in manageable size data packages, which could range from few bytes to many kilo-bytes. Network layer is optional and is used if the implant were to make connections to more than one hosts at the same time, such as mobile phone, another implant, a Galvanically connected external unit and so on. Data Link layer provides the error correction function as illustrated in FIGS. 14 and 15. Finally the Physical Layer provides the actual transmission and reception of the data and instructions, using a scheme that is illustrated in FIG. 4. Depending on the needs and usages, some of the layers may be omitted.

Unless defined otherwise, all technical and scientific terms used herein generally have the same meaning as commonly understood by one of ordinary skill in the relevant art.

The articles “a” and “an” are used herein to refer to one or to more than one (i.e., to at least one) of the grammatical object of the article. For example, “an element” means one element or more than one element.

The term “comprising” includes, but is not limited to, whatever follows the word “comprising.” Use of the term indicates the listed elements are required or mandatory but that other elements are optional and may or may not be present.

The term “consisting of” includes and is limited to whatever follows the phrase “consisting of.” The phrase indicates the limited elements are required or mandatory and that no other elements may be present.

“WiFi” is a wireless network protocol that allows the connection of multiple devices and supports very high data rates and security schemes in addition to longer range compared to other communication protocols, such as Bluetooth. However its power consumption can be higher.

The term Ethernet refers to a wired or wireless communication protocol between at least two devices.

The term “electronics” refers to a set of electronic components including connectors, conductors, active components such as amplifiers, passive components such as resistors, capacitors, inductors and crystals, relays, timer integrated circuits, diodes, wired and wireless communication modules, transistors, and power sources such as batteries and power adapters, motors, solenoids, flow and pressure sensors and temperature sensors.

The term “processor” refers to a digital circuitry that performs tasks in a predetermined order based on its inputs and produces digital outputs, which may be running programs that are written based on chosen algorithms. It can be a microprocessor, or a custom digital circuitry, such as an application specific integrated circuit (ASIC), an embedded controller or a programmable logic array (PLA).

The term “cloud” refers to a common name for centrally hosted systems, public or private, conveniently giving computer services to users or companies.

The above description provides examples of some implementations. Other implementations that are not explicitly described above are also possible, such as implementations based on modifications and/or variations of the features described above. For example, the techniques described above may be implemented in different orders, with the inclusion of one or more additional steps, and/or with the exclusion of one or more of the identified steps. Similarly, the devices, systems and methods described herein may include one or more additional features, may exclude one or more of the identified features, and/or include the identified features combined in a different way than presented above. Features that are described as singular may be implemented as a plurality of such features. Likewise, features that are described as a plurality may be implemented as singular instances of such features. The drawings are intended to be illustrative and may not precisely depict some implementations. Variations in sizing, placement, shapes, angles, curvatures, and/or the positioning of features relative to each other are possible. Accordingly, other implementations are within the scope of the following claims.

1. A method monitoring a physiological signal of a patient, comprising:
sensing, from a subcutaneous implant location with an implantable medical device, a physiological signal of the patient; capturing, by the implantable medical device, the physiological signal and storing data representing the captured physiological signal to memory of the implantable medical device; transmitting, by the implantable medical device for receipt by a device external of the body of the patient, the data representing the captured physiological signal via a Galvanic communications link between the implantable medical device and the device external of the body of the patient, wherein body tissue of the patient serves as a conductive medium for the transmission of the data representing the captured physiological signal.

2. The method of claim 1, wherein the data representing the captured physiological signal is transmitted without using radio frequency telemetry, and without using a radio frequency antenna.

3. The method of claim 1, wherein the data representing the captured physiological signal is transmitted without modulating the data representing the captured physiological signal onto a carrier wave.

4. The method of claim 1, wherein a first electrode and a second electrode are used to capture the physiological signal, and wherein the first electrode and the second electrode are further used to transmit the data representing the captured physiological signal via the Galvanic communications link.

5. The method of claim 1, wherein a first electrode and a second electrode are used to capture the physiological signal, and wherein at least one of the first electrode and the second electrode is further used to transmit the data representing the captured physiological signal via the Galvanic communications link.

6. The method of claim 1, wherein the memory of the implantable medical device comprises a memory device with at least five megabytes of storage capacity.

7. The method of claim 6, wherein a first electrode and a second electrode are used to capture the physiological signal, wherein at least one of the first electrode and the second electrode is further used to transmit the data representing the captured physiological signal via the Galvanic communications link, wherein the data representing the captured physiological signal and transmitted via the Galvanic communications link comprises at least five megabytes of data.

8. The method of claim 7, wherein the data representing the captured physiological signal and transmitted via the Galvanic communications link and comprising at least five megabytes of data is transmitted via the Galvanic communications link over a single communications session.

9. The method of claim 7, wherein the implantable medical device continuously senses and captures the physiological signal over a period of at least four hours, wherein data stored to the memory of the implantable medical device represents the continuously captured physiological signal over the period of at least four hours, and wherein the data transmitted via the Galvanic communications link comprises the data that represents the continuously captured physiological signal over the period of at least four hours.

10. The method of claim 1, further comprising, detecting, by the implantable medical device, an abnormality in the captured physiological signal, and transmitting, via radio frequency telemetry for receipt by a second device external of the body of the patient, data representing the captured physiological signal and the abnormality.

11. (canceled)

12. A system for monitoring a physiological signal of a patient, comprising: an implantable medical device and a device external of the body of the patient; wherein the implantable medical device is configured to:
sense, from a subcutaneous implant location, a physiological signal of the patient; capture the physiological signal and store data representing the captured physiological signal to memory of the implantable medical device; and transmit, for receipt by the device external of the body of the patient, the data representing the captured physiological signal via a Galvanic communications link between the implantable medical device and the device external of the body of the patient, wherein body tissue of the patient serves as a conductive medium for the transmission of the data representing the captured physiological signal; wherein the device external of the body of the patient is configured to:
receive, from the implantable medical device, the data representing the captured physiological signal via the Galvanic communications link between the implantable medical device and the device external of the body of the patient; analyze the data representing the captured physiological signal using an algorithm selected from the group consisting of a machine learning algorithm and an artificial intelligence algorithm; and provide, to the implantable medical device based on a result from the analysis of the data representing the captured physiological signal, a parameter that the implantable medical device can use to adjust an operation on the implantable medical device.

13. (canceled)

14. (canceled)

15. (canceled)

16. (canceled)

17. (canceled)

18. (canceled)

19. (canceled)

20. The system of claim 12, wherein the implantable medical device continuously senses and captures the physiological signal over a period of at least four hours, wherein data stored to the memory of the implantable medical device represents the continuously captured physiological signal over the period of at least four hours, and wherein the data transmitted via the Galvanic communications link comprises the data that represents the continuously captured physiological signal over the period of at least four hours.

21. The system of claim 12, wherein the implantable medical device is further configured to detect an abnormality in the captured physiological signal, and transmit, via radio frequency telemetry for receipt by a second device external of the body of the patient, data representing the captured physiological signal and the abnormality.

22. (canceled)

23. The system of claim 12, wherein the device external of the body of the patient is further configured to receive an input that specifies a clinical event, and wherein the analyzing the data representing the captured physiological signal includes consideration of the received input that specifies a clinical event.

24. The system of claim [24] 12, wherein the input that specifies a clinical event is received from the patient or a physician.

25. (canceled)

26. A system for monitoring a physiological signal of a patient, comprising:
an implantable medical device, a device external of the body of the patient, and a remote computing device; wherein the implantable medical device is configured to:
sense, from a subcutaneous implant location, a physiological signal of the patient; capture the physiological signal and store data representing the captured physiological signal to memory of the implantable medical device; and transmit, for receipt by the device external of the body of the patient, the data representing the captured physiological signal via a Galvanic communications link between the implantable medical device and the device external of the body of the patient, wherein body tissue of the patient serves as a conductive medium for the transmission of the data representing the captured physiological signal; wherein the device external of the body of the patient is configured to:
receive, from the implantable medical device, the data representing the captured physiological signal via the Galvanic communications link between the implantable medical device and the device external of the body of the patient; and transmit, for receipt by the remote computing device, the data representing the captured physiological signal; wherein the remote computing device is configured to:
receive, from the device external of the body of the patient, the data representing the captured physiological signal; analyze the data representing the captured physiological signal using an algorithm selected from the group consisting of a machine learning algorithm and an artificial intelligence algorithm; and provide, to the implantable medical device based on a result from the analysis of the data representing the captured physiological signal, a parameter that the implantable medical device can use to adjust an operation on the implantable medical device.

27. (canceled)

28. (canceled)

29. (canceled)

30. The system of claim 26, wherein the remote computing device is configured to, using the algorithm selected from the group consisting of a machine learning algorithm and an artificial intelligence algorithm, use data associated with a population of patients, and wherein the remote computing device is further configured to provide, to the implantable medical device based on a result from the analysis of the data representing the captured physiological signal and the data associated with the population of patients, a parameter
that the implantable medical device can use to adjust an operation on the implantable medical device.

31. (canceled)

32. (canceled)

33. (canceled)

34. (canceled)

35. (canceled)

36. (canceled)

37. (canceled)

38. (canceled)

39. (canceled)

40. (canceled)

41. The machine learning algorithm of claim 26 is used for the determination of thresholds for triggering alarms based on population data or patient specific data.

42. (canceled)

43. [Thresholds of claims 41 and 42 are used for the generation of alarms that are indicative of worsening conditions that are specific for the disease condition of the patient.]

44. (canceled)

45. Thresholds of claim [s 41 and] 41 are based on one or more of electrical impedance, auscultation, tissue oxygenation, electrogram and acceleration.
